(19) United States
(12) Patent Application Publication
(10) Pub. No.: US 2023/0277110 A1
(43) Pub. Date: Sep. 7, 2023

(54) MICROELECTRODE FOR INSERTION INTO SOFT TISSUE

(71) Applicant: NEURONANO AB, Karlshamn (SE)

(72) Inventor: Jens SCHOUENBORG, Lund (SE)

(21) Appl. No.: 18/004,130

(22) PCT Filed: Jul. 5, 2021
(86) PCT No.: PCT/SE2021/050679

§ 371 Date: Jan. 3, 2023

(30) Foreign Application Priority Data
Jul. 3, 2020 (SE) 2030219-6
Jul. 6, 2020 (SE) 2030220-4

(51) Int. Cl. A61B 5/263; A61N 1/05

(52) U.S. Cl. CPC A61B 5/263; A61N 1/05; A61B 2562/028; A61B 2562/125

(57) ABSTRACT

A microelectrode that is useful for implantation into, or placement adjacent, soft tissue, such as neural tissue, and includes a conductive element having a distal non-insulated portion and a proximal insulated portion. Part of the conductive element is disposed in a casing of electrically insulating non-degradable material, the casing encapsulating the non-insulated portion of the conductive element, and including at least one opening and a first structural component in which the electrically insulated portion of the conductive element can slide in an axial direction.

### FIELD OF THE INVENTION

The present invention relates to a microelectrode configured to be at least partly embedded in soft tissue or at least partly placed adjacent to soft tissue, specifically nervous, endocrine, muscle and connective tissue, comprising an elongated electrically conductive element comprising a distal non-insulated portion and a proximal insulated portion, the non-insulated portion of the conductive element disposed within a casing (envelope) of an electrically insulating non-degradable material, the non-insulated portion of the element encapsulated (surrounded) by the casing forming a distal chamber, in which distal chamber the conductive element can slide (move) in an axial directing without the distal tip the non-insulated conductive element contacting the casing. The casing of the distal chamber has at least one opening and the casing comprises a first structural component in which the electrically insulated portion of the conductive element can slide in an axial direction The invention further encompasses a microelectrode probe, arrays of microelectrodes and/or microelectrode probes and a method for the manufacturing of the microelectrode/microelectrode probe. The various aspects of the present invention are preferably applied for neuromodulation and sensing.

### BACKGROUND AND OBJECTIVES

Implantable microelectrodes and sets of microelectrodes have a wide scope of applications in medicine and veterinary medicine.

A microelectrode implanted into nervous, endocrine or muscle tissue, independent from whether constituting a single implant or pertaining to an implant comprising multiple microelectrodes such as a bundle or array of microelectrodes, requires connection to control device(s) disposed exteriorly of the tissue to be monitored and/or stimulated. This connection is generally provided by thin insulated flexible electrical leads. The leads bridge tissues of various kind and stiffness and thereby become affected by their recurrent displacement relative to each other caused by breathing, heart beets, head and spine movements, position of the brain relative to the skull, and age-related changes. This kind of tissue movement may similarly affect other thin and flexible implants such as microfibers, in particular optical microfibers.

An example of a situation in which movements of tissues relative to each other can be observed is when an electrical lead bridges the skull and the brain via a space comprising dura mater, arachnoid membrane, cerebrospinal fluid, and pia mater. Other examples are leads bridging vertebrae and spinal cord; muscle and adjacent fibrous sheets; peripheral nerve (such as the vagus nerve) and surrounding soft tissue. These movements of tissues relative to each other result in different forces (e.g. shear forces) acting between implanted leads and tissue at their bordering area, which risk causing persistent local inflammation and tissue injury. In addition, shear forces of this kind may affect the position of the active, non-insulated portion (contact) of an implanted microelectrode. Instability of the electrode contact also results in variability of the specific neuronal, endocrine or muscle elements being recorded or stimulated over time, which is especially problematic when monitoring and analyzing long term changes in such signals or when stable long term stimulation is necessary.

A further object of the invention is to provide a microelectrode that can be at least partially embedded (implanted) in soft tissue or at least partly placed adjacent to soft tissue, in particular nervous, muscle or endocrine tissue, and electrically connected with a control apparatus disposed exteriorly of the target tissue, which avoids or at least reduces tissue irritation by movements of tissue abutting it or abutting a lead electrically connecting it with electrode control apparatus disposed outside the tissue of implantation.

Another object of the invention is to prevent or reduce dislocations of an implanted microelectrode contact by forces affecting the lead by which it is electrically connected with electrode control apparatus.

Still another object of the invention is to provide for increased freedom of lateral movement of an implanted microelectrode.

A further object of the invention is to provide a microelectrode probe or an array of such probes for implantation into soft tissue or at least partly placed adjacent to soft tissue, in particular nervous, muscular or endocrine tissue, capable of there being transformed to a microelectrode or an array of microelectrodes by contact with aqueous body fluid.

An additional object of the invention is to provide methods of manufacture for a microelectrode probe and an array of microelectrode probes of the invention.

Other objects will be apparent from the description below. One advantage of the present invention is to avoid or minimize the contact of the element, specifically the non-insulated portion of the element, with adjacent soft tissue. The specific features of the microelectrode enable the microelectrode to accommodate for movements of surrounding soft tissue in all spatial direction, and in particular movements coinciding with the main axis of the microelectrode, while the conductive element (including the non-insulated and insulated portion) is able to move inside the casing without direct contact with surrounding soft tissue.

Once inserted into soft tissue the casing in certain instances may attach to the surrounding soft tissue to a degree that the casing essentially fully adjusts with the surrounding soft tissue. Put slightly different, the casing accommodates and follows to a certain extent for any movement of the surrounding soft tissue

### PRESENTATION OF THE INVENTION

The present invention is based on the insight that direct contact of the conductive electrode (herein referred to as conductive element) of an implanted microelectrode with adjacent soft tissue, in particular nervous tissue but also endocrine tissue, exocrine tissue, muscular tissue and connective tissue, can be avoided by encapsulating the conductive element with a casing of an electrically insulating non-degradable material. The casing provides for a distal compartment and comprises a first structural component in which the electrically insulated portion of the conductive element can slide in an axial direction. An opening in the casing of the distal chamber provides a fluidic electrically conductive bridge between the non-insulated portion of the conductive element and the soft tissue enabling an exchange of ions between the distal chamber and the tissue, wherein the at least one opening is useful for recording and stimulation of electrically excitable cells. The casing is associated with the conductive element such that the distal tip of the non-insulated portion of the conductive element may never touch nor penetrate the casing of the distal chamber.

The detachment of the conductive element from the protective casing and the potential adherence of the casing to the surrounding soft tissue enables the casing to a(n) (significant) extent accommodate for any movement of the surrounding tissue while preserving that the electrode monitors and/or stimulates the same region of the soft tissue over time and that the perpendicular distance (FIG. 24, 104) between the non-insulated distal portion of the element and the opening remains essentially the same over time. Further, the detachment of the conductive element from the protective casing enables the soft tissue surrounding the microelectrode to move without significantly influencing the signal pattern (fingerprint) provided by the microelectrode.

A still further advantage of the present invention is that the casing once inserted into soft tissue may adhere to the soft tissue in a way that minimizes or even essentially prohibits movement of the casing vis-à-vis the surrounding soft tissue. When the soft tissue moves the casing moves with the soft tissue. The decoupling of the casing from the element inside the casing is an important feature for minimizing or essentially prohibiting the movement of the casing in relation to the surrounding soft tissue.

The detachment of the conductive element from the protective casing enables the soft tissue surrounding the microelectrode to move without significantly influencing/altering the perpendicular distance (FIG. 24, 104) of the non-insulated distal portion of the element to the opening(s), since the perpendicular distance will not change significantly when the non-insulating portion of the element slide in an axial direction.

The microelectrode of the present invention preferably provide a high surface area of the non-insulated conductive element while simultaneously providing the stimulation and monitoring of a highly spatially specific region of the soft tissue. The construction also enables the casing to move in relation to the conductive element. Thus, the casing can accommodate for movements of the soft tissue while the non-insulated conductive element is always confined within the casing encapsulating said non-insulated conductive element.

Before presenting the invention in more depth some recurrent terms are described below for facilitating the understanding of the invention.

The terms ‘proximal’ and ‘distal’ are used to specify entities of the different aspects of the invention in relation to optional devices electrically connected to a microelectrode and positioned outside the target tissue (the target tissue being where the stimulation/recordings are to be made). A proximal entity or a proximal part/portion/section/region of an entity is closer (with respect to the length of the connecting microelectrode/lead) to an optional electrical device than a distal entity or a distal part/portion/section/region of an entity. The transition from a proximal part/portion/section/region of an entity to a distal part/portion/section/region of an entity should not be understood as a very specific region, rather, the division of an entity or designation of entities into/as proximal and distal is a means to position such entities in relation to each other. For example, the microelectrode comprises an elongated electrically conductive element having at least a proximal electrically insulated portion and a distal non-insulated portion. The proximal portion of the element is localized closer to an electrical device.

The invention relates, inter alia, to a microelectrode and a microelectrode probe. The microelectrode probe constitutes a version of the microelectrode which is designed to be inserted into soft tissue. Hence, the microelectrode probe comprises certain components providing the probe with sufficient rigidity to enable successful insertion into various soft tissues. Once inserted into soft tissue, certain components of the microelectrode probe dissolves and/or disintegrates upon contact with body fluids transforming the microelectrode gradually into the microelectrode, an in-situ microelectrode.

Common to all aspects of the invention is the microelectrode/microelectrode probe to be at least partially embedded or inserted into soft tissue or at least partially placed adjacent to soft tissue. In its widest definition soft tissue relates to any tissue of any sentient being excluding hard tissue such as bone tissue. More particularly, soft tissue encompasses any soft tissue which provides electric fingerprints which can be monitored and/or any tissue susceptible to electric stimulation. A specifically interesting soft tissue sub-group constitutes nervous tissue, endocrine tissue, muscle tissue and connective tissue. Soft tissue also encompasses hollow fluidic spaces such as ventricles. Nervous tissue is a specifically interesting soft tissue to study and stimulate with the present invention.

Common to all aspects of the invention (microelectrode, proto-microelectrode, microelectrode, arrays) is that the casing of the distal chamber comprises at least one opening. The opening serves multiple purposes. The opening is a prerequisite for the migration of charged particles between the surrounding soft tissue and the distal non-insulated portion of the element.

The microelectrode may be implanted in soft tissue or positioned adjacent to soft tissue. By adjacent should be understood that at least part of the microelectrode is not surrounded by soft tissue. Certain soft tissues may preferably be monitored and/or stimulated by the microelectrode by an adjacent positioning with respect to the soft tissue. Spinal nervous tissue may advantageously be monitored and/or stimulated by positioning the microelectrode adjacent to nervous tissue of the spinal cord.

All aspects of the invention comprise an elongated electrically conductive element. The elongated electrically conductive element can be understood as a thin electrically conductive filament, typically rotationally symmetric, with a diameter or thickness in the range of from about a few μm, e.g. 2 μm, up to about 100 μm. The elongated electrically conductive element (including non-insulated and insulated electrically conductive element) typically has a length of from about 2 mm up to about 1 m. The casing of the microelectrode has typically an elongated form having an axial extension from about 50 μm up to about 20 mm, suitably from about 500 μm up to about 15 mm. The elongated electrically conductive element may comprise several sub-elements. An electrically conductive element may be composed of a plurality of micro or nano filaments which are electrically connected. The conductive element may be designed as a multifilament element, for example a twisted multifilament. A multifilament electrode element usually has a larger surface area than that of a single filament element of the same diameter and thus a lower impedance.

In general, the term ‘flexible’ as contemplated in this invention and in its most generic interpretation should be construed as providing such qualities to the microelectrode and all other aspects of the invention to allow the casing of the microelectrode to at least partly accommodate the movements of the surrounding soft tissue.

The part of the casing forming the proximal compartment may also be denoted proximal casing or casing of the proximal compartment, the part of the casing forming the distal chamber may also be denoted distal casing or casing of the distal chamber.

The term microelectrode as used herein includes at least a conductive element and a casing as described in any of the aspects/embodiment such as comprising a first structural component and at least one opening (in the part of the casing of the distal chamber, the distal chamber encapsulating the non-insulated portion of the conductive element).

In some embodiments the conductive element in disposed in a casing, the casing comprising a first structural component partitioning the casing into a distal chamber and proximal compartment. The terms chamber and compartment have been chosen partly for added clarity. Additionally, the chamber and compartment to an extent serve different purposes and more importantly, the distal chamber embraces in essence the non-insulated portion of the conductive element while the insulated portion of the conductive element is disposed mainly or at least partly in the proximal compartment.

### DISCLOSURE OF THE INVENTION

The present invention relates to a microelectrode, a microelectrode probe, different arrays of microelectrodes and/or microelectrode probes, and a method for the manufacturing of a microelectrode, a microelectrode probe and arrays.

More specifically, the invention related to a microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber having at least one opening providing (after implantation) a fluidic electrically conductive bridge between the non-insulated portion of the conductive element and the soft tissue enabling an exchange of ions between the distal chamber and the tissue, wherein the at least one opening is useful for recording and stimulation of electrically excitable cells, wherein the casing comprises a first structural component in which the electrically insulated portion of the conductive element can slide in an axial direction.

According to an aspect the at least one opening is positioned laterally with respect to the casing of the distal chamber and preferably positioned laterally such that the perpendicular distance between the non-insulated portion of the conductive element and the opening (or openings) during axial movement of the conductive element does not change more than 20%, suitably not more than 15%, preferably not more than 10%,

The non-insulated portion of the conductive element is disposed in a casing of an electrically insulating non-degradable material forming a distal chamber, the casing comprising a first structural component. The first structural component enables the casing to be axially displaced with respect to the conductive element. For the first structural component to slide in axial direction with respect to the insulated portion of the conductive element there should be a void/lumen between the insulated portion of the conductive element and the first structural component. The association of the first structural component with the insulated portion of the conductive element should preferably be configured that the electrical impedance between the non-insulated portion of the conductive element and the soft tissue (adjacent to the at least one opening) is lower than the electrical impedance between the non-insulated portion of the conductive element and the tissue surrounding the proximal part of the proximal compartment or tissue proximally to the first structural component in case there is no proximal compartment.

The invention also relates to a microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion and distal non-insulated portion, at least part of the conductive element being disposed in a casing (envelope) of electrically insulating non-degradable material, wherein the casing comprises a first structural component partitioning the casing (envelope) in a distal chamber and a proximal compartment, wherein the non-insulated portion of the element is encapsulated (surrounded) by the casing (envelope) thereby forming the distal chamber, the casing of the distal chamber comprising at least one opening, wherein the first structural component is configured to slide in axial direction with respect to the electrically insulated portion of the conductive element.

A restriction of charged particles through the lumen between the insulated portion of the conductive element and the first structural component is desirable in the event that for example the distal chamber and proximal compartment of the casing are disposed in different tissues comprising aqueous body fluid differing in composition, and that an exchange of aqueous body fluid between the tissues is to be minimized. This is, for instance, of importance when avoiding communication of cerebrospinal fluid with nervous tissue in the neighborhood of the distal element portion lacking insulation.

If the casing is allowed to follow (adjust to) any movement of the surrounding soft tissue the opening (or openings) of the casing of the distal chamber will essentially over time always be located at nearly the same spatial region in the soft tissue. Hence, the microelectrode of the invention will over time always monitor or stimulate essentially the very same region of the soft tissue. This characteristic is generally of importance for any soft tissue and of particular relevance for nervous tissue such a nervous tissue associated to the brain. The design of the microelectrode significantly improves over prior designs specifically in a dimension that nearly the same spatial region of the soft tissue is monitored and/or stimulated over time and even if soft tissue is displaced.

The opening comprised in the casing of the distal chamber is the opening that provides for an exchange of charged particles, specifically ions, between the non-insulated portion of the conductive element and the soft tissue adjacent to the opening. Thus, the opening provides a fluidic electrically conductive bridge between the non-insulated portion of the conductive element and the soft tissue enabling an exchange of ions between the distal chamber and the tissue useful for recording and stimulation of electrically excitable cells. Electrically excitable cells, such as neurons, are found in any tissue susceptible to electric stimulation including nervous tissue, endocrine tissue, muscle tissue and connective tissue.

The casing comprises at least a first structural component which enables the casing, i.e. the casing defining the distal chamber, to slide axially with respect to the conductive element and specifically with respect to the insulated portion of the conductive element. This first structural element may optionally be an integral part of the casing but can also be provided by an element distinct from the casing. If the microelectrode only comprises a distal chamber the first structural element of the casing suitably constitutes a proximal portion of the casing of the distal chamber narrowing down to a configuration providing a slidable connection with the proximal electrically insulated portion of the conductive element while simultaneously minimizing the exchange of charged particles through any void between the proximal electrically insulated portion of the conductive element and the proximal portion of the casing of the distal chamber

In one embodiment, the casing comprises a first structural element partitioning the casing (envelope) in a distal chamber and a proximal compartment. The distal chamber encapsulates the distal non-insulated portion of the element except for at least one opening.

By encapsulation and distal chamber should be understood that the distal non-insulated portion of the element is essentially electrically isolated from the surrounding tissue by the casing except for the opening or openings in the casing of the distal chamber. Some leak current will often be present over the lumen/void/annular channel between the insulated portion of the conductive element and the first structural component.

Depending on the production method, the first structural element may be an integral part of the casing, alternatively, the first structural element is an element distinct from the casing optionally of a material different from the material of the casing (FIG. 23).

Irrespective if the casing forms only a distal chamber, or, a distal chamber and proximal compartment it is important that the casing can move with respect to the conductive element, specifically in axial direction. In an aspect of the invention, the casing encapsulates the distal non-insulated portion of the element. As the casing needs to be able to move axially with respect to the element the casing should be slidably connected to or engaged with the proximal electrically insulated portion of the element. The part of the casing slidably connected to or engaged with the proximal electrically insulated portion of the element is referred to as the first structural component.

By ‘slidably connected to or engaged with’ should be understood a connection or engagement enabling axial movement while also essentially prohibiting or at least reducing the migration of charged particles (such as electrons and ions) between the distal chamber and the surrounding soft tissue, such as between the distal chamber and proximal compartment or proximal to the first structural components (if the microelectrode lacks a proximal compartment. Put differently, the attachment of the casing to the proximal electrically insulated portion of the element must provide a higher impedance between the distal chamber and proximal compartment (or surrounding soft tissue provided only the casing encapsulates the distal non-insulated portion of the element) over the distance of the attachment while simultaneously enabling an axial movement than between the non-insulated portion of the conductive element and the tissue adjacent to the at least one opening if the casing of the distal chamber.

According to an aspect, the void/lumen/annular channel between the first structural component and the proximal electrically insulated portion of the element, may comprise a composition which is essentially stable over time in tissue fluids and facilitates axial movement of the casing while minimizing migration of charged particles (and thus providing a high impedance over the first structural component). According to an aspect, the composition which is essentially stable over time in tissue fluids and facilitates axial movement of the casing while minimizing migration of charged particles may be a composition facilitating the movement of the first structural element with respect to the outermost layer, particularly a composition comprising any one of lipids, hyaluronic acid, silicones (such as silicone oil or silicone grease) and a polymer of monosaccharides such as glucose and combinations thereof.

Some embodiments, such as microelectrodes, microelectrode probes and arrays, comprise a biocompatible material providing rigidity to the probe when dry for insertion into soft tissue and dissolvable or degradable in aqueous body fluids. The term rigidity when dry should be interpreted as a dryness causing the material to crack under load (radial or axial load) instead of bending.

Useful biocompatible materials providing sufficient rigidity to the probe when dry for insertion into soft tissue and dissolvable or degradable in aqueous body fluids. The biocompatible materials, also referred to as matrices, are suitably chosen from protein-based (proteinaceous) materials, carbohydrate-based materials, and polyethylene glycols of various molecular weights. A suitable protein-based matrix material is gelatin typically derived from collagen. A suitable carbohydrate-based matrix material is glucose. The biocompatible matrix material may be selected from gelatin, glucose and polyethylene glycol.

According to all embodiments the insulation surrounding the insulated portion of the conductive element is non-degradable in body fluids. The insulation material may be chosen from any materials of the casing.

Should the first structural element be distinct to the casing, it is important that the attachment of the first structural element to the casing prohibits migration of charged particles. The material of the first structural element must also be electrically insulating.

According to an aspect, the first structural component has an extension in axial direction of at least from about 5 μm up to about 10 mm, preferably from about 5 μm up to about 3 mm.

According to an aspect, at least part of the electrically insulated portion is localized within the distal chamber.

According to a further aspect, a lumen/void (enabling axial movements) is provided between the first structural component and the electrically insulated portion of the conductive element.

The lumen/void may also be contemplated as an annular channel formed between the first structural component and the electrically insulated portion of the conductive element.

It is preferred that the lumen/void/annular channel between the first structural component and the electrically insulated element restricts radial movements of the conductive element with respect to the distal casing and that the impedance of this lumen/void is higher than the impedance over the opening(s) in the distal casing.

According to a further aspect, the proximal portion of the distal chamber narrows down, exhibiting an annular structure forming the first structural component, in which first structural component and the electrically insulated portion of the conductive element can slide in an axial direction.

It is important that the entire casing can move, typically in axial direction, with respect to the conductive element.

According to an aspect, the innermost material(s) of the casing and/or the first structural components and/or the outermost material of the proximal electrically insulated portion of the element is/are (each) selected to reduce friction.

The first structural component may be any shape of the casing or non-casing component enabling the casing to move in axial direction with respect to the insulated conductive element and provide an impedance over the first structural component in relation to the impedance between the non-insulated portion of the conductive element and the opening(s) in the distal casing which renders useful recordings and stimulation of electrically excitable cells (neurons) adjacent to the at least one opening in the casing of the distal chamber.

According to an aspect, the electrical impedance between the non-insulated portion of the conductive element and the soft tissue (adjacent to the at least one opening) is lower than the electrical impedance between the non-insulated portion of the conductive element and the tissue surrounding the proximal part of the proximal compartment or tissue proximally to the first structural component (in case there is no proximal compartment).

According to a further aspect, the electrical impedance between the non-insulated portion of the conductive element and the soft tissue (adjacent to the at least one opening) is at least 5 times lower, preferably at least 25 times lower, preferably at least 100 times lower, than the electrical impedance between the non-insulated portion of the conductive element and the tissue surrounding the proximal part of the proximal compartment or tissue proximally to the first structural component.

According to yet a further aspect, the first structural component and the proximal electrically insulated portion of the conductive element forms an annular channel, wherein the electrical impedance over the channel (when filled with body fluids) is at least 5 times higher, preferably at least 25 times higher, preferably at least 100 times higher than the electrical impedance of the opening or openings in the distal casing and wherein the channel enables the first structural element to slide with respect to the conductive element in an axial direction.

It is preferred that the axial movement of the non-insulated portion of the conductive element does not significantly influence the radial positioning within the distal casing. Preferably, perpendicular distance (FIG. 24, 104) between the non-insulated portion of the conductive element and the at least one opening in the casing of the distal chamber remains essentially the same during axial movements of the casing relative to the conductive element, optionally less than 20%.

A variation of the distance of the non-insulated portion of the conductive element will inevitably lead to a variation in the distance to the monitored tissue (adjacent an opening) which will have an impact on the fingerprint of the recorded signals. A variation of the distance may induce amplitude variance of recorded signals interfering with the ability to distinguish signals from unique cells.

According to an aspect the distal chamber comprises a second structural component configured to reducing radial movement of the non-insulated portion of the conductive element relative to the distal casing, while also being configured to enable an axial movement of the non-isolated conductive element with respect to the second structural component. This second structural component may form part of the casing, thus being an integral part of the casing. However, the second structural component may also be distinct from the casing. For example, the second structural component may be of Teflon, attached to the casing and comprising a central channel enabling the non-insulated portion of the conductive element to axially move.

According to an aspect, the material of the second structural component is distinct from the material of the casing being at least partly attached to the casing and configured to be slidably connected to or engaged with the non-isolated conductive element.

The casing of the distal chamber must have at least one opening providing (after implantation) a fluidic electrically conductive bridge between the non-insulated portion of the conductive element and the soft tissue enabling an exchange of ions between the distal chamber and the tissue, wherein the at least one opening is useful for recording and stimulation of electrically excitable cells.

According to an aspect, wherein the at least one opening has an area of at least about 1 μm². Preferably, an individual opening of the casing of the distal chamber has an area from about 1 μm² up to about 150000 μm² or more.

Furthermore, the opening should preferably have the characteristics of prohibiting the blockage of the opening or openings by tissue. It has been observed that glial cells can cover small opening and then to some extent isolate the interior of the distal chamber from the surrounding neurons. A preferred range of the area of an opening is from about 20 μm² up to about 2000 μm², suitably from about 100 to about 1500 μm².

According to a further aspect, the casing of the distal chamber comprises a plurality of openings in the distal casing.

According to yet a further aspect, the maximum number of openings of the distal chamber is given by the maximum number of openings not significantly compromising the structural rigidity/conformation of the distal casing.

The proximal insulated portion of the conductive element may comprise a segment which facilitates flexing in axial and radial direction.

According to an aspect, the distal portion of the casing of the distal chamber has a three-dimensional shape narrowing in distal direction. Such a three-dimensional shape may be spherical, paraboloid (elliptically paraboloid), or conical.

It is preferred that the casing accommodates for movements of the soft tissue while the conductive element can move with respect to the casing.

According to an aspect, the casing comprises means for increasing friction between the casing and the adjacent soft tissue. Preferably, the means for increasing friction is selected from micro- or nano-fibers attached to the outermost surface of the casing.

Thus, according to an aspect, the friction between the casing and the adjacent soft tissue is higher that the friction between the innermost material of the casing and/or the first structural component and/or the outermost material of the proximal electrically insulated portion of the element.

A further aspect is that the outermost material and/or outermost surface structure of the casing is selected to increase friction against the soft tissue.

According to yet a further aspect, the casing comprises two layers of materials an inner layer and outer layer, wherein the material of the inner layer is different from the material of the outer layer or wherein the surface structure of the inner layer is different from surface structure of the outer layer.

The microelectrode may comprise an engagement element configured to reversibly engage with an elongated rigid pin, such as a needle, the rigid pin being configured to insert the microelectrode into the soft tissue or placing the microelectrode adjacent to soft tissue. The engagement element is suitably positioned at the distal tip of the microelectrode, but can also be positioned along the distal casing. Thus, the engagement element may be positioned at a distal portion of the casing, such as the distal portion of the distal casing, including the distal tip of the distal casing. If the microelectrode comprises an engagement element the microelectrode may be inserted into soft tissue or positioned adjacent to soft tissue by way of a rigid pin reversibly engaging with the engagement element, the rigid pin (such as a needle) forming part of an apparatus for inserting microelectrodes into soft tissue as disclosed by e.g. US 2020/0086111 A1. If a microelectrode is inserted by the use of a rigid pin (reversibly engaging with an engagement element of the microelectrode) there is less of a need that the microelectrode per se exhibit an intrinsic rigidity. Thus, a microelectrode comprising an engagement element may at least partly dispense with any material providing the microelectrode with rigidity, such as a biocompatible material providing sufficient rigidity to the probe when dry for insertion into soft tissue and dissolvable or degradable in aqueous body fluids.

The engagement element may constitute a loop or comprise a net. According to an aspect the engagement element may also constitute non-degradable or degradable micro- or nano-fibers, the micro- or nano-fibers being adhesively attached to the microelectrode, typically attached to the casing, specifically to the distal section of the casing, such as the distal casing. The microfibers may be any of the micro- or nano-fibers disclosed herein.

According to an embodiment the microelectrode comprises a biocompatible material providing sufficient rigidity to the probe/microelectrode when dry for insertion into soft tissue and dissolvable or degradable in aqueous body fluids. The material imparting structural rigidity to the microelectrode is typically found in the distal chamber and optionally also preset in the proximal compartment or around at least part of the insulated portion of the conductive element.

The microelectrode may also be disposed in a material providing sufficient rigidity to the probe when dry for insertion into soft tissue and dissolvable or degradable in aqueous body fluids. One may contemplate a microelectrode comprising a casing and a distal chamber and optionally a proximal compartment, where the distal chamber and the optional proximal compartment do not comprise a biocompatible material providing sufficient rigidity, yet, the microelectrode being disposed in a biocompatible material providing sufficient rigidity.

According to yet a further aspect, the casing has a rotationally symmetric shape, suitably cylindrical shape. Preferably the radial extension of the casing of the distal chamber and at least part of the casing of the proximal compartment (typically the distal portion of the proximal compartment) is similar or essentially same. Suitably, the radial extension over the distal chamber and at least part of the proximal compartment does not differ more than 20%, typically not more than 10%.

According to yet a further aspect, diameter of the proximal compartment widens in a proximal direction.

A microelectrode comprising biocompatible material increasing rigidity is herein also referred to as a microelectrode probe.

A further aspect of the invention relates to arrays, such as first and second arrays, of microelectrodes and/or microelectrode probes. In its widest definition an array is characterized by at least two microelectrodes/microelectrode probes, the array structure capable of being implanted into soft tissue or positioning adjacent to soft tissue, a number of microelectrodes/probes of even first arrays disposed in a set spatial conformation without essentially changing the disposition during insertion. An array is typically provided by embedding microelectrodes and/or microelectrode probes, or first arrays in an array matrix. An array may constitute a plurality of individual microelectrodes and/or microelectrode probes or first arrays arranged in various three-dimensional shapes.

Arrays of microelectrodes/probes adhesively attached to micro- or nano-fibers are denotes as first arrays.

Second arrays denote an assembly of at least two microelectrode/probes or first arrays which are embedded in an array matrix. Thus, a second array may also comprise a first array.

The individual microelectrodes/probes may be arranged in any conceivable spatial configuration of first and second arrays. Configuration may embrace axial sections, each section comprising a plurality of individual microelectrode having same of different spatial configurations.

According to an embodiment of an array (first and second arrays), the microelectrodes are disposed substantially in parallel.

If an array comprises a plurality of microelectrodes, such as three or more, it is preferred that the axis of one microelectrode essentially coincides with the main axis of the array with remaining microelectrodes positioned radially around the axis of the array. Furthermore, the distal ends of the microelectrode may be disposed essentially in a plane perpendicular to the axes of the microelectrodes (FIG. 20).

The arrays may have a configuration that associates microelectrodes/probes with each other. One type of association limits movements of microelectrodes with respect to each other. The adhesive attachment of microelectrodes with micro- or nano fibers of the first arrays is a means to limit movements of microelectrodes to each other. An array configured to associate microelectrodes may also be referred to as a bundle of microelectrodes. E.g. the microelectrodes, such as the casings of the microelectrodes, may be adhesively attached to each other. Alternatively, the microelectrodes of an array are arranged to move independently when inserted into soft tissue. In this variant the microelectrodes are spatially positioned only by the array matrix.

According to an embodiment the array comprises an array cover. The array matrix may be configured to extend to distal face of the array cover.

According to a further embodiment the array matrix may be in part be covered by an array casing of any of the electrically insulating material presented herein.

According to yet a further embodiment, the array may comprise a further outer array matrix.

Additionally, the invention also encompasses an array of microelectrodes the microelectrodes being adhesively attached to microfibers. Suitably, the microfibers are capable over time to essentially maintain the mutual spatial positioning of the microfibers of the array when the array is positioned adjacent to soft tissue or embedded in soft tissue.

A further aspect of the invention relates to a microelectrode probe. The microelectrode probe comprises features enabling the successful implantation of the probe by the insertion into soft tissue. Thus, the microelectrode probe comprises, with respect to the microelectrode, components providing the probe with sufficient rigidity to be inserted into soft tissue. Alternatively, the microelectrode may be transformed into a probe by altering the rigidity of materials of the microelectrode, typically the casing, enabling the insertion of the microelectrode into soft tissue for example by altering the temperature of the materials transiently.

Also, the distal non-insulated portion of the element is entirely localized in the distal chamber and entirely encapsulated by the casing except for at least one opening.

According to an embodiment the distal section of the distal chamber distal chamber narrows in distal direction. Preferably, the distal section of the distal chamber is of the same material as the casing. The distal section of the distal chamber provides for a sliding movement of the conductive element in the distal direction.

An elongated electrically conductive element comprising at least a proximal electrically insulated portion and a non-insulated portion is at least in part disposed within a casing of an electrically insulating material. An important feature of all aspects of the invention is the encapsulation of the distal non-insulated portion of the element by a casing of an electrically insulating non-degradable material thus forming a distal chamber. According to an embodiment, the casing comprises a first structural element partitioning the casing (envelope) in a distal and proximal compartment, the distal chamber encapsulating the distal non-insulated portion of the element except for an opening in the casing. The casing serves several purposes. The casing is configured to enable it to move in axial direction with respect to the element.

Furthermore, the casing is configured to partition/divide the casing into a proximal and distal chamber by way of a first structural element. The first structural element may constitute an integral part of the casing. Alternatively, the first structural element may constitute a separate entity with respect to the casing. In the former, the first structural element shares the same material as the casing. In the latter, the first structural element may be of a different material than the casing. It is preferred that the microelectrode is configured such that the physical contact of the conductive element with the casing is minimized specifically with the distal non-insulated portion of the element. Apparent lateral movements of the conductive element with respect to the casing tend to be a function of the distance from the tubular structure of the first structural element. Hence, the distal tip of the non-insulated element tends to have a more pronounced lateral movement with respect to the casing than the part of the element closer to the tubular structure.

In principle, the casing can have any form as long as the conductive element can be disposed within the casing. It may be favorable that the casing is rotationally symmetric in an effort to avoid the element to contact the casing. According to one embodiment, the casing is rotationally symmetric typically with respect to a central axis normally coinciding with the main axis of the element. The three-dimensional form of the casing may have an impact on the rigidity of the casing. Hence, the rigidity of the casing can be modulated not only by way of the choice of casing material but also the choice of three-dimensional form of the casing. One preferred three-dimensional form of the casing is the cylindric form. Preferably, the element is disposed in a casing of cylindric form where the element essentially coincides with the main axis of the cylindrically formed casing.

As alluded to above, the casing is the prime facilitator for letting surrounding soft tissue not significantly interfere with the conductive element in general and specifically the distal non-insulated portion of the element present in the distal chamber. The casing may be attached to a first structural component which may have the form of a tubular structure enabling charged particles to pass between the proximal compartment and distal chamber through the lumen/void between the conductive element (outermost layer of the element) and the first structural component. Should the first structural component be an entity distinct from the casing, the tubular structure must abut and/or adhere to the casing. The first structural component suitably comprises an arrangement such as an elongated tube configured to provide a lumen/void between the element, in particular the electrically insulated portion of the element and the tubular structure. The volume of the lumen/void should enable a movement of the first structural component with respect to the conductive element, specifically an axial movement of the tubular structure.

According to an embodiment, the void/lumen (defined by the space between the proximal electrically insulated portion of the element and the first structural element) has an extension in axial direction satisfying as least one of the following criteria: a) allowing the first structural element (e.g. tubular structure) to move with respect to the element, b) allowing the tubular structure to move with respect to the element while simultaneously centralizing the casing with respect to the axis of the element, c) providing a difference in terms of the electric impedance emergent between the proximal compartment and distal chamber on the one hand and the electric impedance emergent between the distal non-insulated portion of the element and the (surrounding) soft tissue one the other hand.

The electric impedance emerging between the proximal compartment and distal chamber is to an extent a function of the extension of the void/lumen in axial direction and the volume of the void/lumen between the first structural element (tubular structure) and the element. At a given extension of the first structural element a reduction of the volume of the void/lumen will increase the electric impedance between the proximal compartment and distal chamber.

The greater the axial extension of the void/lumen the higher the impedance at a given area of the void/lumen in a plane perpendicular to the axis of the element (and implicitly the microelectrode). An increase in axial extension of the void/lumen also tends to increase the friction between the element and the tubular structure. The axial extension of the void/lumen must satisfy the criteria of providing a sufficiently high electrical impedance while enabling the tubular structure to slide with respect to the element.

According to an embodiment, the friction between the casing and the surrounding soft tissue is higher, preferably significantly higher, than the friction between the conductive element and the casing (including first structural element). The difference in friction is as least such that useful patterns of data can be extracted from the microelectrode. Specifically, the difference in friction is as least such that useful patterns of data can be extracted from the same region of the soft tissue over time.

According to an embodiment, the casing (or first structural element) is configured to provide a higher electric impedance (between the proximal compartment and distal chamber) than between the distal non-insulated portion of the element and the soft tissue. More specifically, the electric impedance provided by the tubular structure is suitably at least about 5 times higher, preferably at least about 25 times higher, preferably at least about 1000 times higher than the impedance between the distal non-insulated portion of the element and the soft tissue.

Generally, the axial extension of the distal casing, defining a distal chamber in particular the void distal to the non-insulated conductive element and the axial extension of the 1^{st} structural element is partly correlated to the normally occurring displacements of the soft tissue abutting the openings in the distal casings in relation to a proximal connection, typically localized in the skull or vertebra. Thus, the extension of the void/lumen of the casing (or the tubular structure) is dependent on the spatial movements of the respective tissue. The extension of the void/lumen in axial direction may broadly range from at least about 300 μm up to about 20 mm. Notably, the extension is much smaller for smaller animals than for larger animals and can also be smaller for soft tissue not moving much in relation to the tissue surrounding the proximal connection.

The materials of the casing and the outermost material surrounding the element may be selected with the aim of facilitating the movement of the tubular structure with respect to the element in axial direction.

It is important that the material of the casing is electrically insulating and non-degradable. For the microelectrode to function properly, it is important that the casing is not degraded or dissolved over time, i.e. the life span of the microelectrode once positioned into soft tissue.

The outermost material surrounding the conductive element at the location of the sliding first structural component may constitute the electrical insulation per se.

Furthermore, the void/lumen between the inner surface of the first structural component and the outermost material surrounding the conductive element may comprise a composition (medium) facilitating the axial movement of the tubular structure with respect to the conductive element. Such a composition may be selected from lipids, silicones and compositions comprising hyaluronic acid and a polymer of disaccharides or a composition mimicking the characteristics of synovial fluid.

According to one embodiment the distal end of the distal chamber is provided as a distal end cap. The cap has typically a shape shielding the distal tip of the non-insulated from interacting with the surrounding soft tissue. Furthermore, the distal end of the distal chamber should also have a shape and length that allows the conductive element to move axially without penetrating the cap. The distal end of the distal chamber has suitably a shape narrowing in distal direction. The distal end of the distal chamber may be pointy (sharp/acute) or dome shaped. The distal end of the distal chamber may have a spherical shape.

A further embodiment of the microelectrode comprises a second structural component configured to minimize lateral (radial) movements of the distal non-insulated portion of the element. The second structural component should also allow the element to move in axial direction. Several secondary structural components may be positioned within the distal chamber for positioning the element centrally. The second structural component may be integrated with the casing and adhere to the inner surface of the casing or optionally being made of the same material as the casing. Alternatively, the second structural component may be distinct from the casing preferably made of materials other than casing materials. Lateral movements of the distal non-insulated portion of the element with respect to the casing and specifically with respect to the opening(s) may alter the shortest distance between the distal non-insulated portion of the element and the soft tissue and, hence, have an implication for the impedance between the distal non-insulated portion of the element and the soft tissue which in turn may affect the measurement/stimulation. The casing is made of an electrically insulating non-degradable material. The casing material should be able to accommodate (move with) any type of spatial movement of the surrounding soft tissue.

The dimensions of the microelectrode are such that materials may be used for the casing which are stiff at macroscopic dimensions but become sufficiently flexible at the dimensions of the microelectrode. Hence, various crystalline materials may be contemplated as casing materials, such as crystalline materials comprising silicon dioxide such as any material referred to as glass. According to a preferred embodiment, the electrically insulating material is an electrically insulating non-degradable flexible polymeric material. Suitable electrically insulating non-degradable flexible polymeric materials are polymeric materials which can be disposed by dip coating, spray coating, vapor deposition or casting or any combination thereof.

Suitable electrically insulating flexible non-degradable polymeric materials include polytetrafluoreten (Teflon), Parylene C, polyurethanes, polyethylenes and polymers comprising a backbone of recurring aromatic moieties such as aromatic moieties comprising an aromatic six-membered ring structure exemplified by para benzenediyl moieties. Preferred polymeric materials are polymers obtained by the polymerization of para-xylene. Hydrogen atoms of the polymers comprising a backbone of recurring aromatic moieties may be substituted by various functional groups. Parylenes are a preferred class of electrically insulating flexible polymeric materials sharing the characteristics of polymers comprising a backbone of recurring aromatic moieties such as aromatic moieties comprising an aromatic six-membered ring structure exemplified by bara benzenediyl moieties. The polymeric materials may be chosen from Parylenen C and Parylene M.

All materials of the microelectrode that are in contact with tissue, such as electrically insulating materials, must be biocompatible.

According to a further embodiment the proximal electrically insulated portion of the conductive element is configured to accommodate for spatial movements of the soft tissue. The proximal electrically insulated portion of the conductive element may comprise at least one section facilitating flexing of the element particularly flexing in a direction partly coinciding with the main axis of the element (microelectrode) and/or a section facilitating bending in radial direction. This flexing section of the element may be localized proximally to the proximal compartment between the proximal compartment and a holder. Alternatively, the flexing section may be localized within the proximal compartment, i.e. fully disposed in the casing of the proximal compartment. The section facilitating flexing enables the proximal electrically insulated portion of the element to be elongated by at least about 10% (based on the length of the proximal insulated portion in equilibrium state), at least about 20%, at least about 50% and preferably at least about 100%. The section facilitating elongation (flexing) of the electrically insulated portion of the element can be chosen form any of the following forms: spiral form, zig-zag-form, meandering form, or any combination of the forms.

The material of the electrically conductive element can be any electrically conductive material fulfilling the characteristics of a microelectrode for implantation into soft tissue, specifically neural, endocrine or muscular tissue. A variety of metals are suitable, but also conductive non-metal materials. Suitable materials are metals or mixtures of metals which reduce or even omit oxidation in the tissue surrounding the microelectrode, including platinum, iridium, gold, wolfram, stainless steel, and alloys thereof. More specifically, suitable metals of the element are selected from platinum, iridium, gold, wolfram, stainless steel and alloys thereof. Conductive non-metal materials include various conductive polymers and carbon-containing materials such as graphene, graphite and carbon nanotubes.

The element can be of a single metal or comprise two or more portions of different metals. Alternatively, the element can comprise two or more ultra-thin metallic wires. The thickness of the one or more wires is preferably from about 100 nm to 1 μm or 10 μm or even 100 μm. The two or more ultra-thin wires may be entangled such that the surface area is maximized.

The section of the electrically insulated portion of the element extending proximally of the proximal compartment can be of a material or of materials different from that or those of the portion disposed in the proximal compartment and distal chamber. The non-insulated portion of the element present within the distal chamber may exhibit sections of the surface with a higher surface area than the average surface area of the non-insulated portion of the element within the distal chamber. Suitably, the sections(s) exhibiting a higher surface area is(are) localized in the vicinity of the opening(s) of the distal chamber. The non-insulated portion of the element present in the distal chamber may also comprise rugged sections or comprise protrusions near the opening(s). The rugged sections or protrusions are in the micro or nano scale.

As recited in the claims the distal non-insulated portion of the element is entirely localized within the distal chamber.

According to an embodiment, during operation of the microelectrode, the most distal section of the insulated proximal portion of the element should preferably always be comprised in the distal chamber. The casing should suitably be positioned in relation to the conductive element such that the casing always fully embraces the non-insulated portion of the conductive element irrespective of axial movement of the casing. Also, the conductive element should be originally positioned in the casing such that the distal tip of the non-insulating portion of the conductive element never reaches the casing of the distal chamber. Alternatively, the microelectrode may have a means which limits the axial movement of either the casing or the conductive element such that the distal tip of the non-insulated conductive element may never contact the casing or puncture the casing.

The casing (e.g. first structural element) may be positioned initially at a location with respect to the insulated proximal portion of the element that the probability that the first structural element will to an extent leave the insulated portion of the element (and whole or partially slide over the non-insulated portion) is minimal or virtually non-existent.

The number of openings depends to a degree on the volume of the distal chamber, type of material(s) of the casing and the mode of operation of the microelectrode. When using the microelectrode for stimulation soft tissue it may be preferable to have a higher total area of openings (higher number of openings) than when using the microelectrode for soft tissue monitoring purposes. Should the microelectrode operate both in stimulation and monitoring mode the total area of openings (number of openings) should preferably be within a range satisfying both the needs of stimulation and monitoring modes. The upper number of openings is to an extent governed by the structural rigidity of the distal chamber (of the casing encapsulating the distal chamber), the area of one opening preferably being within a range of from about 20 μm² up to about 150000 μm² or more.

Implanted microelectrodes may need to be removed from the surrounding tissue. In order to facilitate the removal the microelectrode may comprise a flexible filament securely attached to the microelectrode at a location facilitating the removal. The proximal portion of such flexible filament should be located such that the filament is easily retrievable without undue irritation of any tissue.

A further aspect of the invention relates to a microelectrode probe. As already alluded to above the microelectrode probe constitutes a version of the microelectrode which is designed to be inserted into soft tissue. Hence, the microelectrode probe comprises certain components providing the probe with sufficient rigidity to be successfully inserted into various soft tissues. Once inserted into soft tissue, certain components of the microelectrode probe dissolves and/or disintegrates upon contact with body fluids transforming the microelectrode gradually into the microelectrode, an in-situ microelectrode.

It should be noted that all embodiments and structural features of the microelectrode configured to be embedded into soft tissue are equally relevant to the microelectrode probe.

The microelectrode probe comprises matrices of biocompatible materials providing sufficient rigidity to the probe when dry for insertion into soft tissue and dissolvable or degradable in aqueous body fluids. The matrices are suitably chosen from protein-based (proteinaceous) materials, carbohydrate-based materials, and polyethylene glycols of various molecular weights. A suitable protein-based matrix material is gelatin typically derived from collagen. A suitable carbohydrate-based matrix material is glucose. The biocompatible matrix material may be selected from gelatin, glucose and polyethylene glycol. The distal chamber which is encapsulated by the casing should preferably comprise a matrix material that does not significantly increase its volume when dissolving in aqueous fluids. The matrix of the distal chamber may have the characteristics that the volume increase of the matrix when absorbing an aqueous fluid is offset by the dissolution/degradation of the matrix.

Matrix materials increasing their volume when absorbing an aqueous fluid may preferably be used for embedding matrices or for cavities/compartments provided by casing materials sufficiently flexible for not undergoing structural damages during matrix volume expansion.

Any of the variants/embodiments of the microelectrode presented (specifically above) may be provided as a microelectrode probe.

One variant of the microelectrode comprises a casing encapsulating the distal non-insulated portion of the element forming the distal chamber but lacks a proximal compartment. The microelectrode probe of this ‘one-compartment’ variant of the microelectrode comprises a distal matrix. It is furthermore preferred to have a proximal matrix around part of the proximal insulated portion of the element. Preferably, this proximal matrix has a spatial radial extension similar to the spatial radial extension of the distal chamber. The proximal matrix may enclose a rigid pin/bar used when inserting the microelectrode. It is preferred that the pin has the same main axis as the distal chamber.

The microelectrode probe for implantation by insertion into soft tissue, in particular nervous, endocrine and muscle tissue, comprises an elongated electrically conductive element having at least a proximal electrically insulated portion and distal non-insulated portion, at least part of the element being disposed in a casing of an electrically insulating non-degradable material, where the distal non-insulated portion of the element is encapsulated by a casing of an electrically insulating non-degradable material forming a distal chamber, the distal chamber having at least one opening, wherein the casing is slidably attached to the proximal electrically insulated portion of the distal chamber; where the distal chamber comprises a distal matrix comprising a biocompatible materials providing sufficient rigidity to the probe when dry for insertion into soft tissue and dissolvable or degradable in aqueous body fluids; and wherein the distal chamber comprises at least one opening through the casing.

A further aspect related to a microelectrode probe for implantation by insertion into soft tissue, in particular nervous, endocrine and muscle tissue, comprising an elongated electrically conductive element having at least a proximal electrically insulated portion and distal non-insulated portion, at least part of the element being disposed in a casing of an electrically non-degradable insulating material, the casing comprising a first structural element partitioning the casing (envelope) in a distal and proximal compartment; the structural element being slidably attached to the proximal electrically insulated portion of the element, wherein the distal casing comprises at least one opening, wherein at least part of the proximal electrically insulated portion is localized within the distal chamber, and wherein the distal and proximal compartments comprise distal and proximal matrices comprising biocompatible material providing sufficient rigidity to the probe when dry for insertion into soft tissue and dissolvable or degradable in aqueous body fluids.

The proximal and distal matrices may not be of the same material. Furthermore, the matrices, be it proximal and distal matrices or any other matrix of the probe or array, may comprise substances biologically active substances such as pharmacologically active substances and gene constructs. According to an embodiment, the distal matrix may comprise biologically active substances.

Biologically active substances are suitably selected from anti-inflammatory substances, neurotrofic substances, sedatives, transmitter substances such as glutamate, glycine, GABA, dopamine, noradrenalin, and acetylcholine. The pharmacologically active substances are suitably comprised within the distal chamber such that these substances can be released through opening(s) in the distal chamber. The biologically active substance may during the manufacturing of the microelectrode probe be added to any of the matrices, such as distal, proximal, embedding, array embedding matrix, either to just one matrix, some of them or all of them. According to an embodiment, the biologically active substance is added to the surface of the distal matrix and/or is comprised in the distal matrix. Also, the biologically active substance may be applied on the element, specifically to the distal non-insulated portion of the element located within the distal chamber.

According to an embodiment, the microelectrode probe may also comprise a further matrix embedding the microelectrode featuring distal and optionally proximal compartments comprising matrices. Such matrices embedding the microelectrode are referred to as embedding matrices.

If the microelectrode probe comprising proximal and distal chambers is not embedded in an embedding matrix it is preferred to apply a further matrix in the space between the proximal compartment and distal chamber referred to as an intermediate matrix. The radial extension of the intermediate matrix suitably follows the radial extensions of the proximal compartment and distal chamber.

The microelectrode or the microelectrode probe may also comprise an element holder. The element holder preferably comprises or consists of a stiff material and comprises a distal face and a proximal face. It is preferred that a proximal terminal section of the proximal insulated portion of the element penetrate the element holder from the distal to the proximal face. It is preferred for the element holder to comprise a cylindrical tube of smaller diameter than that of the element holder, in particular of a diameter equal to or smaller than the diameter of the bore in a bone at which the element holder is to be mounted, the tube extending from a distal face of the element holder in a distal direction. The tube is of same material as the holder or of a different material and is stable against degradation by aqueous body fluid.

A further embodiment is related to an array of microelectrodes comprising microfibers, the microelectrodes being adhesively attached to microfibers. Suitably, the microfibers are capable over time to essentially maintain the mutual spatial positioning of the microelectrodes of the array when the microelectrode array is positioned adjacent to soft tissue or embedded in soft tissue. The microfibers are preferably biodegradable. The array of microelectrodes comprising microfibers may be disposed in a rigid matrix of biocompatible material providing sufficient rigidity to the array when dry for insertion into soft tissue and dissolvable or degradable in aqueous body fluids. The matrix of biocompatible material providing sufficient rigidity is preferably dissolvable/degradable in body fluids at a rate substantially superior to the rate of the degradation of the microfibers. The rigid matrix of biocompatible material providing sufficient rigidity has suitably a degradation/dissolution which is superior to the rate of microfiber degradation by a factor of 2, or 5, or 10 or 20, in particular of 100 or more.

Microfibers for use in the invention are preferably degradable by hydrolysis, in particular by enzymatically enhanced hydrolysis. It is particularly preferred for microfibers of the invention to be used in form of non-woven nano- or microfiber aggregates. Non-woven microfiber aggregates consist of irregularly intertwined microfibers and may comprise microfibers attached to each other in an irregular manner such as by attachment caused by local melting and/or by gluing with a biocompatible glue.

The time for positional stabilization by integration with the nano- and microfibers may range from a few days, such as 2 or 5 or ten days to a couple of weeks, such as 2 or 5 weeks, and occasionally even a few months to years. Degradable microfibers of this kind are known in the art, such as microfibers of polylactide and poly(lactide-co-glycolide), polyvinyl acetate and polyvinyl alcohol and their cross-linked modifications, the molecular weight of which can be varied to provide for suitable rates of degradation. Other microfibers for use in the invention are natural and synthetic proteinaceous microfibers, such as fibrin microfibers, collagen microfibers, laminin microfibers, fibronectin microfibers, cross-linked gelatin microfibers, silk microfibers produced from aqueous protein solutions as disclosed by Viney C and Bell F I (Curr Opin Solid State Mater Sci. 8 (2005) 164-169) but also inorganic microfibers such phosphate glass microfibers, for instance P4oNa2oCa16Mg24 phosphate glass microfibers disclosed in U.S. Pat. No. 8,182,496 B2. Microfibers of the invention are in the micro- or nanometer diameter range. Particularly preferred are electrospun nano- and microfibers and electrospinning is a preferred method for producing microfibers of the invention. It is within the ambit of the invention to provide the device with a net of fibrin microfibers by electrospinning fibrinogen, such as by the method of S R Perumcherry et al. disclosed in Tissue Eng Part C Methods 17; (2011) 1121-30 or with a net of poly(lactide-co-glycolide)/fibrin microfibers such as one disclosed by Perumcherry et al. in Tissue Eng Part A 19; 7-8(2012) 849-859. A self-assembling fibrin net can also be produced by applying an aqueous solution of fibrinogen and thrombin rich in calcium directly to a microelectrode, then cross-linking the microfibers by applying an aqueous solution of plasma transglutaminase and/or factor XIII on the newly formed net for crosslinking.

It is preferred for a microfiber to be selected from proteinaceous microfiber and polyester fiber. Preferred fibrous materials include those based on poly(lactide), poly(lactide-co-glycolide), poly(glycolide), electrospun albumin, mucus material rich in glycoprotein. A particularly preferred kind of microfibers are electro-spun microfibers. According to preferred aspect of the invention the microfibers form a non-woven irregular structure. It is preferred for a microfiber to be adhesively attached to a microelectrode and to one or more other microfibers. Preferably the microfibers are disposed along 50% or more of the axial extension of a microelectrode. Microfibers for use in the invention can be of a resilient or a non-resilient material.

Another aspect of the invention relates to processes for the manufacturing the microelectrodes and microelectrode probes. Dependent on the tubular structure two different manufacturing processes are presented. FIGS. 6 to 16 disclose several manufacturing stages for the manufacturing of a microelectrode/microelectrode probe where the tubular structure forms an integral part of the casing. FIG. 22 illustrates one stage of the manufacturing process where the tubular structure is not integrated with the casing but is separate from the casing.

The invention encompasses a method for manufacturing the microelectrode, microelectrode probe or array, comprising:
providing an elongated electrically conductive element,
covering a proximal portion of the element with an electrically insulating layer thereby providing a proximal electrically insulated portion and a distal non-insulated portion of the conductive element;
forming a distal matrix dissolvable or degradable in aqueous body fluids extending axially around, and optionally extending in a distal direction from, the distal non-insulated portion of the conductive element;
applying a sliding facilitating composition to an section of the insulated element proximally with respect to the distal matrix and distally with respect to an optional proximal matrix which sliding facilitating composition facilitating the axial movement of a first layer of electrically insulating non-degradable material with respect to the insulating layer of the conductive element, said medium optionally providing for a sufficient void/lumen between the insulating layer of the conductive element and first layer of electrically insulating non-degradable material;
optionally forming a proximal matrix extending axially around at least part of the proximal electrically insulated portion of the conductive element;
covering the distal matrix and at least part of the proximal electrically insulated portion of the conductive element with a first layer of electrically insulating non-degradable material, thereby providing a casing encapsulating the distal non-insulated portion of the element forming a distal chamber and a first structural element
cutting the non-insulated portion of the conductive element (preferably a part of the the non-insulated portion of the conductive element) and first layer of electrically insulating non-degradable material near the distal end of the distal matrix comprising the distal non-insulated portion of the electrically conductive element, thereby providing a distal opening of the distal chamber.
applying a further distal tip matrix distally to the distal opening,
covering the tip matrix and at least part of the first layer with a second layer of electrically insulating non-degradable material and at least part of the first layer, thereby forming a distal end cap part forming part of the casing of the distal chamber
wherein the distal and optionally proximal matrices provide structural support to the microelectrode or probe when dry for insertion into soft tissue and;
and wherein at least an opening through the first layer and optionally second layer of the casing of the distal chamber is provided suitably by laser evaporation and optionally followed by laser milling.

A further variant of the method for manufacturing the microelectrode, microelectrode probe, or array as disclosed herein comprises:
providing an elongated electrically conductive element;
covering a proximal portion of the element with an electrically insulating layer thereby providing a proximal electrically insulated portion and a distal non-insulated portion of the conductive element;
forming a distal matrix dissolvable or degradable in aqueous body fluid extending axially around, and optionally extending in a distal direction from, the distal non-insulated portion of the conductive element;
forming a proximal matrix extending axially around at least part of the proximal electrically insulated portion of the conductive element and thereby forming an intermediate section of the insulated conductive element with an axial extension, the intermediate section positioned proximally to the distal matrix and distally to the proximal matrix not covered by the distal and proximal matrices;
applying a thin (up to about 5 um) layer of a first intermediate matrix and/or sliding facilitating composition to the intermediate section of the insulated element facilitating the axial movement of a first layer of electrically insulating non-degradable material with respect to the insulating layer of the conducing element, said first intermediate matrix and/or composition providing for a sufficient void/lumen (annular channel) between the electrically insulated portion of the conductive element and the first layer of electrically insulating non-degradable material;
covering distal, proximal matrices and the intermediate section of the proximal electrically insulated portion of the element, the intermediate section comprising an intermediate matrix and/or sliding facilitating composition, with a first layer of electrically insulating non-degradable material, thereby providing a casing comprising a distal chamber, a first structural element and a proximal compartment;
optionally providing a second intermediate matrix on the first layer of electrically insulating non-degradable material in the constriction in radial direction of the first layer between the distal chamber and proximal compartment;
cutting (part of) the distal non-insulated portion of the electrically conductive element and the first layer of electrically insulating material near the distal end of the distal matrix (distal end of the distal chamber), thereby providing a distal opening of the distal chamber;
applying a further distal tip matrix distally to the distal opening;
covering the distal tip matrix and at least part of the first layer with a second layer of electrically insulating material thereby forming a distal end cap forming part of the casing of the distal chamber;
and removing the first layer and optionally second layer at a circumferential annular zone of the proximal matrix;
wherein the distal matrix, distal tip matrix, proximal matrix and optionally first and second intermediate matrices are of a biocompatible material providing sufficient rigidity to the probe when dry for insertion into soft tissue and dissolvable or degradable in aqueous body fluids;
and wherein at least one opening is provided through the first and optionally second layers of the casing of the distal chamber suitably by evaporation and optionally followed by laser milling.

Still a further embodiment of a method for manufacturing the microelectrode comprising:
providing an elongated electrically conducting element,
covering a proximal portion of the element with an electrically insulating layer thereby providing a proximal electrically insulated portion and a distal non-insulated portion of the element;
providing a first structural element configured to enable an axial movement with respect to the proximal electrically insulated portion of the conductive element;
positioning the first structural element around the proximal electrically insulated portion of the element, suitably at a certain axial distance from the distal non-insulated portion of the conductive element;
applying a proximal matrix dissolvable or degradable in aqueous body fluids around the proximal electrically insulated portion of the conductive element, the proximal matrix extending from the proximal face of the first structural element in proximal direction;
applying a distal matrix dissolvable or degradable in aqueous body fluids around the distal non-insulated portion of the conductive element extending from the distal face of the first structural element in distal direction, and extending in a distal direction from the distal non-insulated portion of the conductive element
applying a first layer of electrically insulating non-degradable material on the proximal and distal matrices and the circumference of the first structural element, thereby forming a casing comprising a distal chamber and a proximal compartment; and wherein at least one opening is provided through the first layer of the casing of the distal chamber suitably by evaporation and optionally followed by laser milling.

According to an aspect, the proximal matrix widens in a proximal direction.

### SHORT DESCRIPTION OF THE FIGURES

FIG. 1 A region of neural tissue for implantation of a microelectrode probe of the invention, in a section perpendicular to a bone protecting the region

FIG. 2 The region of FIG. 1 after providing a circular hole in the bone, in the same section

FIG. 3a A schematic representation of a microelectrode probe of the invention in an axial section

FIG. 3 An electrode according to FIG. 3a immediately upon implantation

FIG. 4 A microelectrode of the invention with a plurality of openings through the distal chamber

FIG. 5 A microelectrode of the invention with a distal chamber but without a proximal compartment proximally to the distal chamber

FIG. 5a A microelectrode of the invention featuring a tubular structure distinct from the casing.

FIG. 5b A microelectrode of the invention featuring a tubular structure distinct from the casing further comprising a structural element within distal chamber

FIG. 6-14 A process for the manufacturing of a microelectrode probe of the invention showing consecutive pre-stages to the microelectrode probe illustrated in FIG. 15

FIG. 15 Microelectrode probe of the invention in axial direction

FIG. 16 A variety of a microelectrode of the invention comprising an embedding matrix

FIG. 17 A microelectrode probe of the invention implanted in neural tissue prior to the dissolution of embedding matrix and proximal and distal matrices

FIG. 18 A proto microelectrode of the invention implanted into neural tissue in a state of partial dissolution of the embedding matrix and in a stage of transformation to a microelectrode of the invention

FIG. 19 A microelectrode of the invention formed in situ (in situ microelectrode) from the microelectrode probe of FIG. 17

FIG. 19a A microelectrode of the invention formed in situ (in situ microelectrode) from the microelectrode probe of FIG. 17. The casing has accommodated for spatial movement of the surrounding soft tissue.

FIG. 20 An array of four microelectrode probes of the invention

FIG. 21 A tubular cross section of the array through the distal chambers of the microelectrode probes

FIG. 22 Half mold with tubular structure of a manufacturing step for producing a microelectrode featuring a tubular structure distinct from the casing

FIG. 23 Tubular structure comprised in variants of the microelectrode

FIG. 24 A variant of the microelectrode of the invention where the radial extension of the casing of the distal compartment is only marginally wider that the radial extension of the insulated portion of the conductive element.

FIG. 25 An array of microelectrodes. The individual microelectrodes are held together by a web of micro- or nano-fibers.

FIG. 26 A microelectrode comprising an engaging element. The casing also exhibits micro- or nano-fibers increasing the friction of the casing with respect to the surrounding soft tissue.

Several embodiments of the invention are describes in more detail below. The embodiments should not be construed as to limit the general concept of the invention.

### DESCRIPTION OF SOME EMBODIMENTS

Implantation and Tissue Environment Principles.

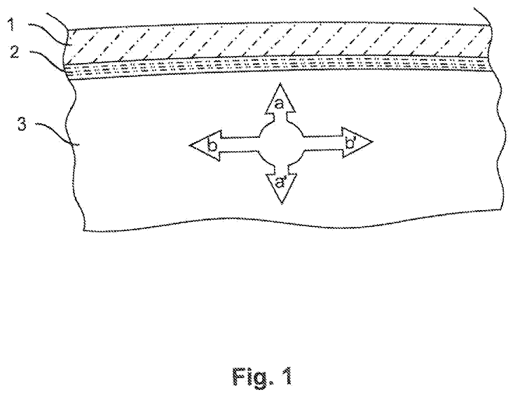

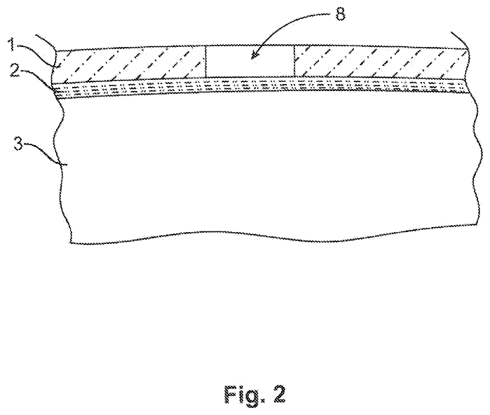

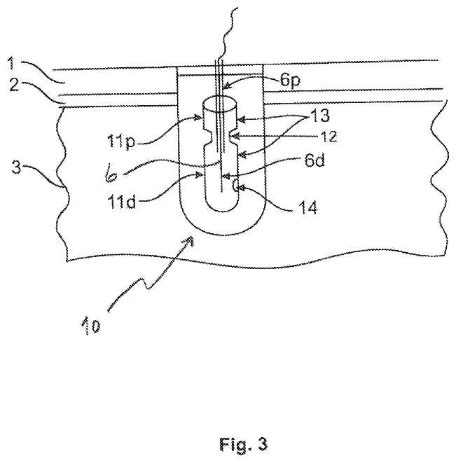

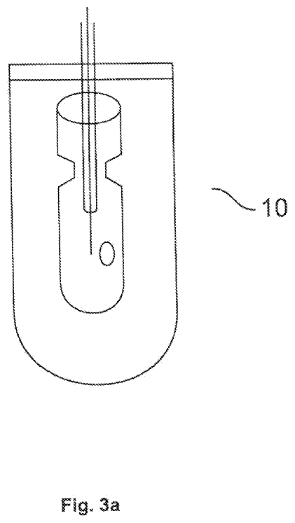

FIGS. 1, 2, 3a and 3 illustrate schematically the intersection of a skull without a microelectrode (FIG. 1 and FIG. 2), an implanted microelectrode probe into neural tissue (FIG. 3) and a microelectrode probe (FIG. 3a). The neural tissue (3) here is brain tissue, protected by the skull bone (1) from which it is separated by a thin layer (2) comprising several sub-layers, such as the dura mater, the arachnoid mater, the pia mater and cerebrospinal fluid. The neural tissue (3) is prone to spatial displacement in respect of the skull bone (1) by movements of the head, the displacement schematically depicted in direction parallel with the skull bone (1) (arrows b, b′) and perpendicular direction (arrows a, a′). Tissues (2) intermediate between the skull bone 1 and brain tissue 3 are similarly displaced but not necessarily to the same extent.

Prior to implantation of a device according to the invention access to a desired position of the brain is provided by drilling a circular hole (8) in the skull (FIG. 2).

In the next step a device of the invention, such as the microelectrode probe (10) of the invention of FIG. 3 a or a microelectrode probe array, is inserted through the hole (8) into brain tissue (3) (FIG. 3). Upon implantation the microelectrode probe (10) is transformed into a microelectrode (in situ microelectrode) of the invention by contact with aqueous body fluid. The fully functional in situ electrode is formed once the matrix materials have completely dissolved or been degraded. The microelectrode probe (10) comprises a cover (7) anchored in the skull bone at the hole (8) protecting the skull bone and soft tissue. The microelectrode (10) comprises a metallic or other electrically conductive element (6) attached to and penetrating the cover (7), which extends from the proximal face of the cover (7) for electrical communication with a microelectrode control unit (not shown) disposed extracorporeally or implanted under the skin. A proximal portion of the element (6p) is electrically insulated while a distal portion of the conductive element (6p) is non-insulated. A first structural component (12) divides the casing (13) into a proximal compartment (11p) and a distal chamber (11d). The distal chamber is encapsulated by the casing further comprising an opening (14) enabling an electric current to flow between the distal non-insulated portion of the conductive element (6d) and the neural tissue (3). In this microelectrode the first structural element is integrated with casing. The first structural element forms an integral part of the casing. Hence, the casing and the first structural element share the same material. The casing, i.e first structural element, is slidably connected to the proximal insulated portion of the conductive element (6p).

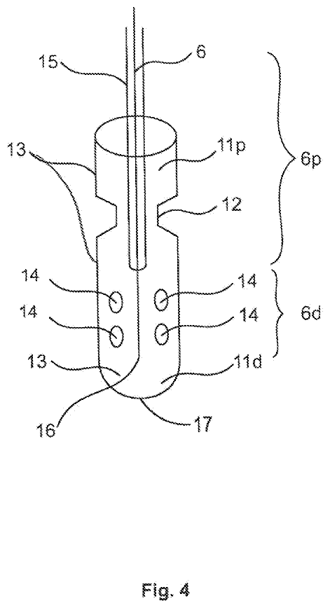

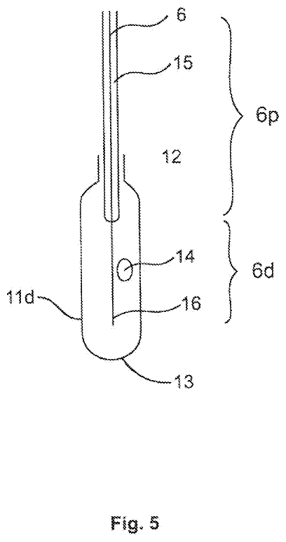

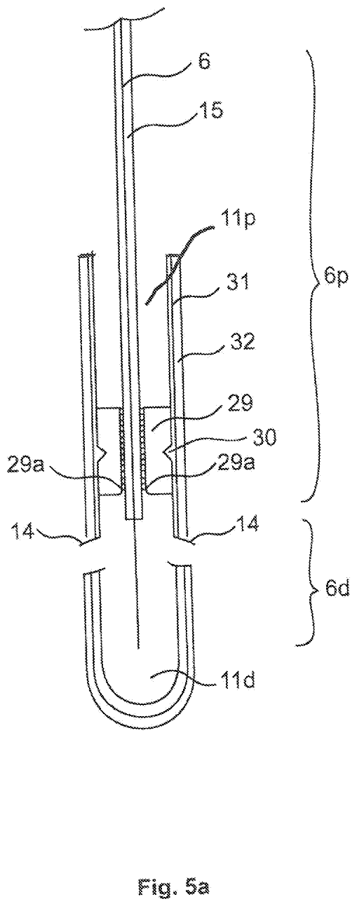

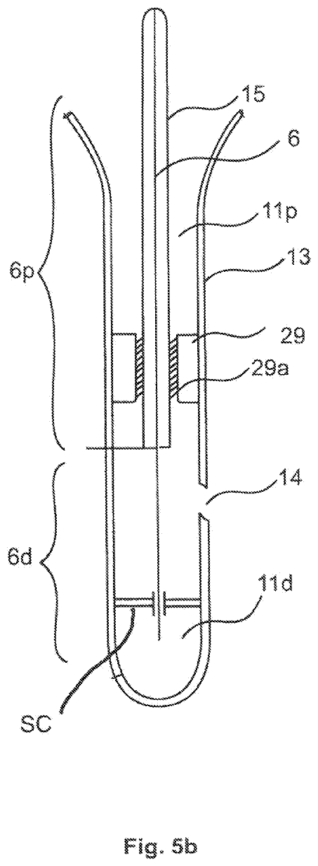

FIGS. 4, 5, 5a and 5b show three variants of the microelectrode as configurated after complete dissolution of matrices.

FIG. 4 shows a variant of the microelectrode as configurated after complete dissolution of the matrices of biocompatible material dissolvable or degradable in aqueous body fluids. This variant comprises a proximal (11p) compartment and a distal chamber (11d). Between the proximal compartment and distal chamber a first structural component (12) is present embracing the proximal insulated portion (6p) of the conductive element (6). As seen in FIG. 4 the casing (13) encapsulates the distal chamber (11d). The first structural component (12) embracing the insulated portion of the conductive element (6p) is slidably attached to the outermost layer of the proximal insulated portion of the conductive element. Here, the outermost layer is equivalent to the insulating layer (15) of the proximal portion of the conductive element (6p). Instead of one opening the distal chamber has four openings (14). All four openings are axially positioned such that the perpendicular distance of the distal non-insulated portion of the conductive element (6d) to the openings remains essentially constant when the conductive element (6), i.e. distal non-insulated portion of the conductive element (6d) and proximal insulated portion of the conductive element (6p), moves with respect to the first structural component (12) which coincides with the movement of the conductive element with respect to the casing encapsulating the distal chamber. The distal tip (16) of the non-insulated conductive element should have enough travel distance in axial direction that the tip never penetrates the casing of the distal end cap (17) of the casing of the distal chamber (11d).

FIG. 5 depicts a microelectrode variant comprising only a distal chamber (11d) encapsulation the distal non-insulated portion (6d) of the conductive element (6). The casing gradually transforms into a first structural element (integrated tubular structure) (12), the first structural element (12) being slidably attached to the proximal electrically insulated portion (6p) of the conductive element. The proximal insulated portion (6p) of the conductive element has an electrically insulating layer (15). The distal compartment comprises an opening (14). The opening (14) is located axially such that the perpendicular distance of the opening (14) with respect to the non-insulated portion of the conductive element (6d) remains essentially constant even if the conductive element (6), i.e. the non-insulated portion of the conductive element (6d), moves in axial direction.

FIG. 5a shows a variant of the microelectrode comprising a first structural element (29) which does not form part of the casing (material) (31), (32). The first structural element which may be of Teflon® comprises a channel which accommodates the proximal insulated portion of the conductive element (6p). The first structural element features a recess (30) which may reach around the whole circumference of the first structural element. The recess secures the attachment of the casing (31) to the first structural element. The void/lumen (annular channel) (29a) between the proximal insulated portion of the conductive element and the first structural element is sufficient for the proximal insulated portion of the element to slide with respect to the first structural element. The casing comprising 1^{st} layer (31) and 2^{nd} layer (32) can be of Parylenen C. Alternatively, 1^{st} (31) and 2^{nd} layers (32) can be made of different material. The 2^{nd} layer (32) may be of a material different from the material of the 1^{st} layer. Said 2^{nd} layer (32) may be a layer which exhibits increased friction with respect to the surrounding soft tissue compered to the material of the 1^{st} layer. Alternatively, or additionally, the outer surface of the 2^{nd} layer may exhibit a friction inducing surface structure.

FIG. 5b illustrates a variant sharing many of the design elements of the microelectrode of FIG. 5a with a difference that a second structural component (SC) is situated within the distal compartment (11d). The second structural component stabilizes the distal non-insulated portion (6d) of the element in radial (lateral) direction. Even if the soft tissue surrounding the microelectrode would move extensively displacing the casing extensively with respect to the element the second structural component (SC) stabilizes the radial movement of the distal non-insulated portion (6d) of the element resulting that the perpendicular distance between the distal non-insulated portion 6d of the element and the opening 14 remains similar over time.

Manufacture of a Microelectrode of the Invention.

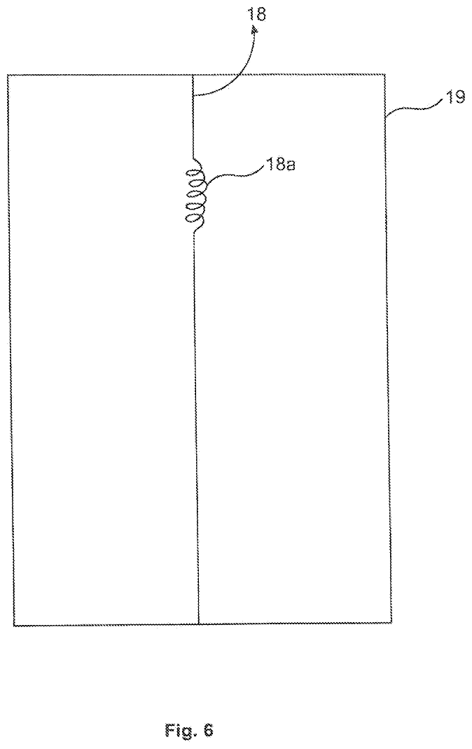

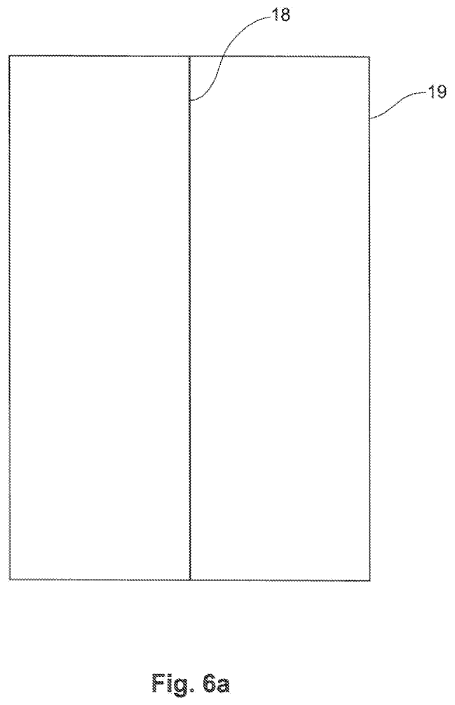

FIG. 6 to 16 show several consecutive steps of one method of manufacturing of a microelectrode probe featuring a first structural component integrated with the casing.

A metallic filament (conductive element) (18) is fastened at both ends to a frame (19).

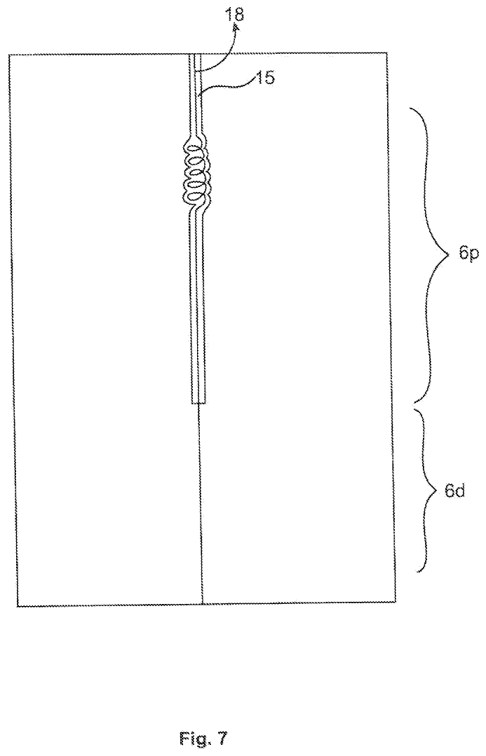

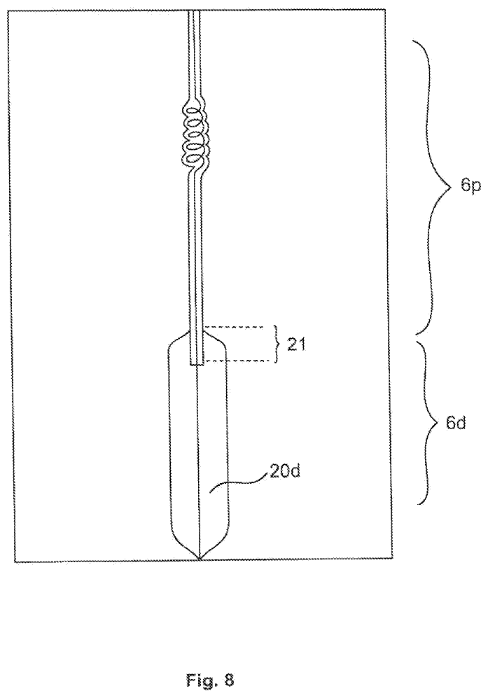

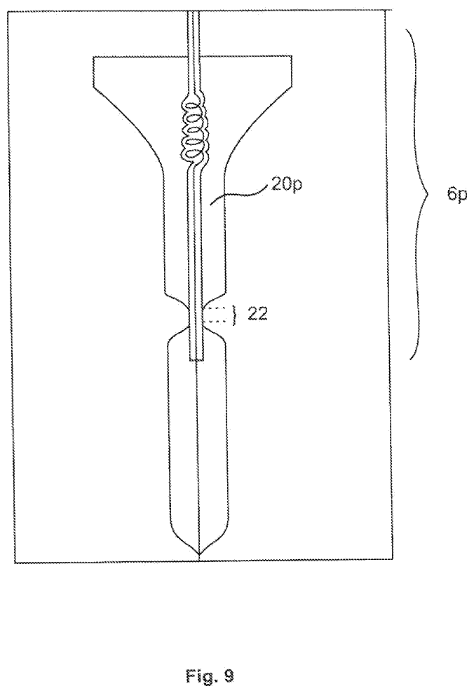

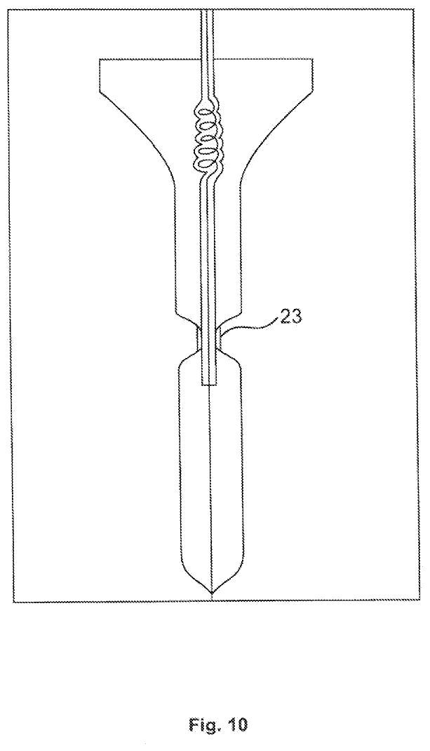

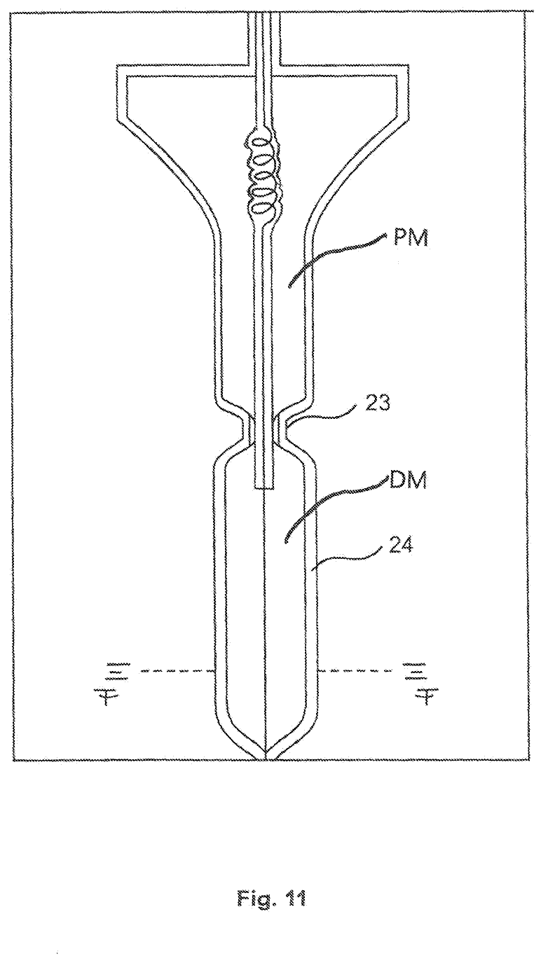

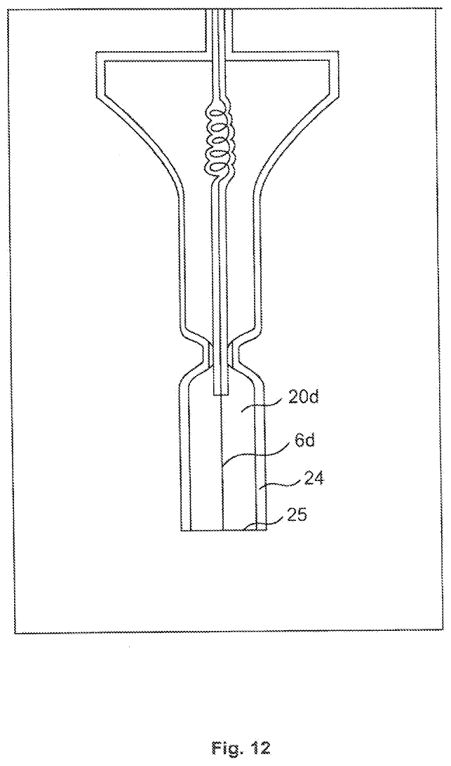

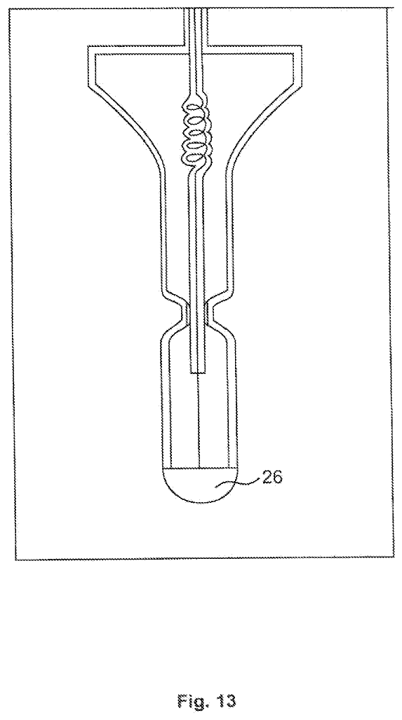

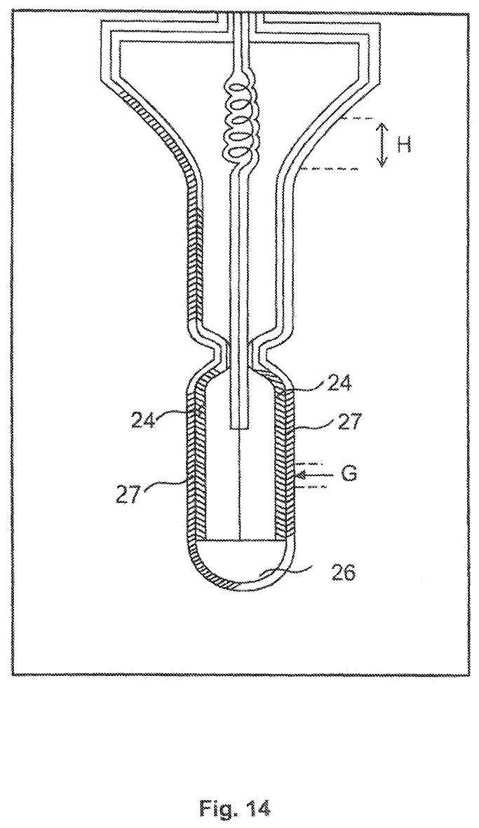

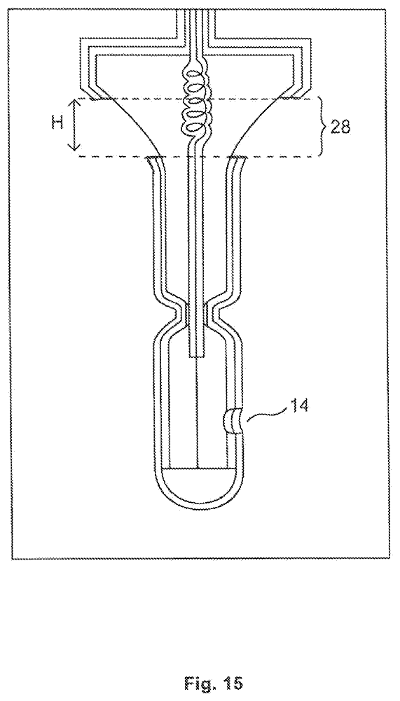

The metallic filament comprises a section (18a) which specifically enables the filament to flex in axial direction (FIG. 6). FIG. 6a shows a frame (19) with a conductive element (18) which does not comprise a section enabling the element to flex in axial direction. In a subsequent step a portion (6p) of the filament is covered with an electrically insulating non-degradable material (15), thereby forming the proximal insulated portion of the conductive element (6p). A distal portion of the conductive element (18) is not covered (6d) thereby providing the prerequisite for forming a distal non-insulated portion of the conductive element. Next (FIG. 8) a distal matrix (20d) is formed radially around the distal portion of the non-insulated conductive element and part of the distal section (21) of the proximal insulated portion of the element. It is important that the matrix also covers part of the proximal insulated portion of the element (21). In FIG. 9 a proximal matrix (20p) is applied radially around part of the proximal insulated portion of the element (6p). An intermediate section (22) remains uncovered by matrix or preferably a thin layer of matrix of biocompatible material is dissolvable or degradable in aqueous body fluids or other composition/substance, such as a composition facilitating the movement of the first structural element with respect to the insulated portion of the conductive element (23) (FIG. 10) is applied to the intermediate section around the element defining a void/lumen (annular channel) (23) between a 1^{st} layer of electrically insulating non-degradable material (such as parylenen) (24) (FIG. 11). If a matrix or composition/substance is applied around the intermediate section of the proximal insulated portion of the conductive element such composition/substance may also facilitate axial movement of the casing (first structural element) and/or modulate the electric impedance between the proximal and distal compartments. FIG. 11 shows a 1^{st} layer of electrically insulating non-degradable material (24) applied to the distal matrix (DM), intermediate section, and proximal matrix (PM). In a further step (FIG. 12) the non-insulated conductive element (6d), distal matrix (20d) and 1^{st} layer (24) are cut radially at a section F-F (FIG. 11) whereby a distal opening (25) is formed which in a subsequent step (FIG. 13) is covered by a distal cap (tip) matrix (26) of a spherical form. FIG. 14 depicts a 2^{nd} layer of electrically insulating non-degradable material (27) covering the distal cap matrix (26) and 1^{st} electrically insulating layer of electrically insulating non-degradable material (24). An opening (14) (FIG. 15) is provided through the casing encapsulating the distal chamber at an allocation G (FIG. 14). Furthermore, 1^{st} and 2^{nd} electrically insulating layers (24, 27) are removed around a circumferential band of height H forming an annular zone (28, FIG. 15) not covered by electrically insulating non-degradable material. The opening may be accomplished by laser evaporation and optionally followed by laser milling evaporation (FIG. 15).

The positioning and axial extent of the circumferential band may vary dependent on the types of tissues to be penetrated by the microelectrode probe.

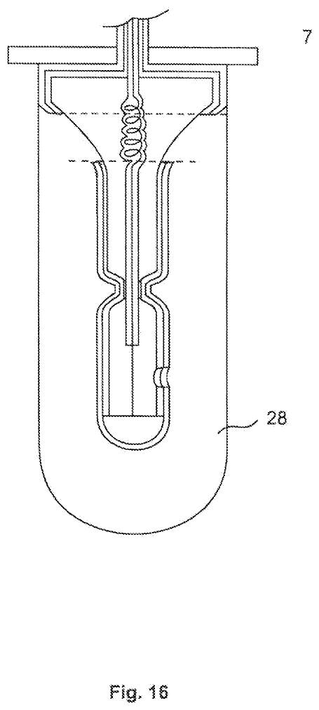

The opening (or openings) is/are preferably positioned axially with respect to the non-insulated element such that the (perpendicular) distance between the non-insulated element and the opening(s) remain(s) essentially similar when the non-insulated element moves axially. In a final step (FIG. 16) the proto microelectrode is covered by an embedding matrix (28) of biocompatible material dissolvable or degradable in aqueous body fluids. The embedding matrix can be formed by spray coating gelatin in a dry atmosphere. The microelectrodes of FIGS. 15 and 16 are both suitable to be inserted into soft tissue. Hence, FIGS. 15 and 16 present microelectrode probes. FIG. 16 also illustrates a cover (7) attached to the proximal face of the casing, the casing formed by 1^{st} and 2^{nd} electrically insulating layer of an electrically insulating non-degradable material. 1^{st} and 2^{nd} electrically insulating layer are preferably of Parylene C.

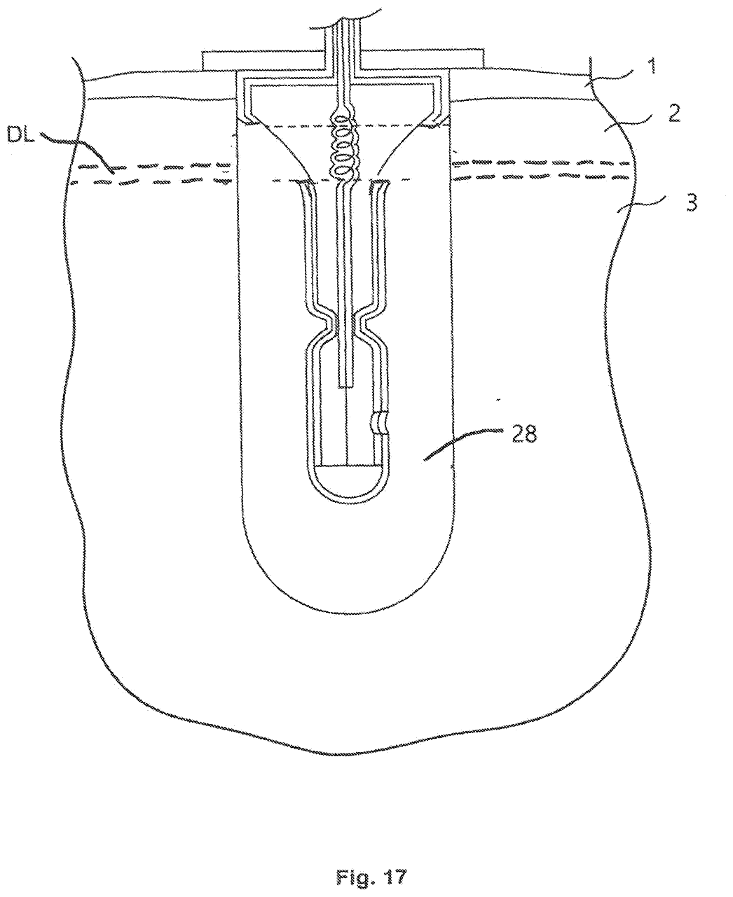

FIG. 17 to 19 depict the microelectrode probe in various states after introduction into soft tissue (3) such as brain tissue. FIG. 17 presents the microelectrode probe immediately after inserted into brain tissue (3) through the skull bone (1) and tissue (2) intermediate between the skull bone such as dura mater, arachnoid membrane, cerebrospinal fluid, and pia mater (1) and brain tissue (3) (neuronal tissue) and prior to the dissolution of matrices. The two discontinued lines DL illustrate tissue regions which may have different characteristics as to e.g. the tendency for spatial movement (2 and 3).

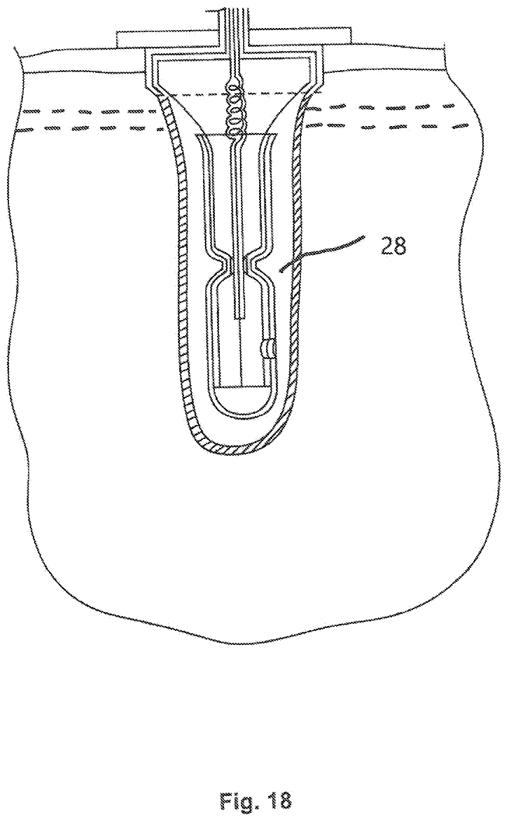

FIG. 18 indicates a partial dissolution of the embedding matrix (28).

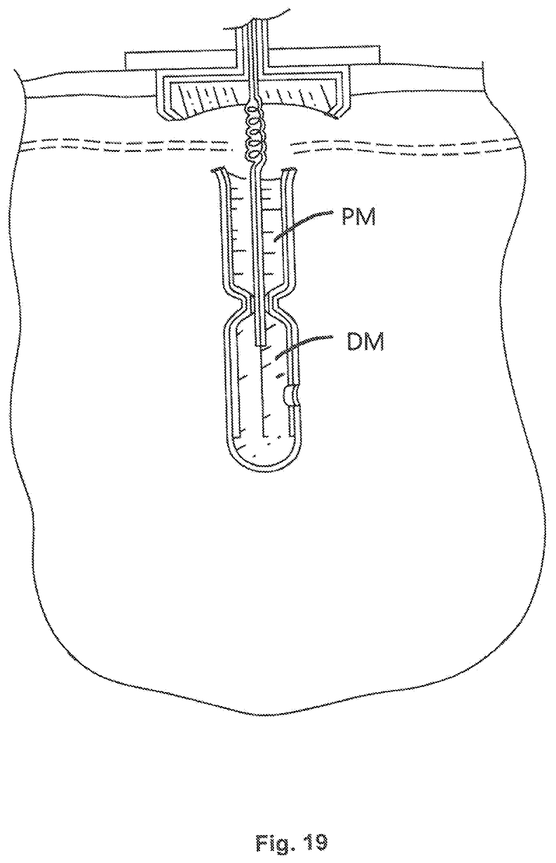

FIG. 19 illustrates a state of the microelectrode probe after complete dissolution of the embedding matrix and partial dissolution of distal (DM) and proximal (PM) matrices.

FIG. 19a is an example of a configuration of a microelectrode after complete dissolution of all matrices showing spatial movement of surrounding soft tissue. The casing (13) which may comprise 1^{st} and 2^{nd} electrically insulating layers of electrically insulating non-degradable material has attached (associated) to the surrounding soft tissue at a degree for being able to accommodate to the spatial movements of the soft tissue. The microelectrode also comprises a structural component SC stabilizing the movement of the non-insulated distal portion of the element (6d). The structural component SC is configured such that the distal portion of the element 6d can move in axial direction without much friction, yet, stabilizing the distal proportion sufficiently radially (laterally) that the (perpendicular) distance between distal non-insulated portion of element with respect to the opening 14 remains essentially same. Once the casing has attached to the surrounding tissue the opening of the casing communicates with essentially the same region of the soft tissue over time even when the soft tissue is moving.

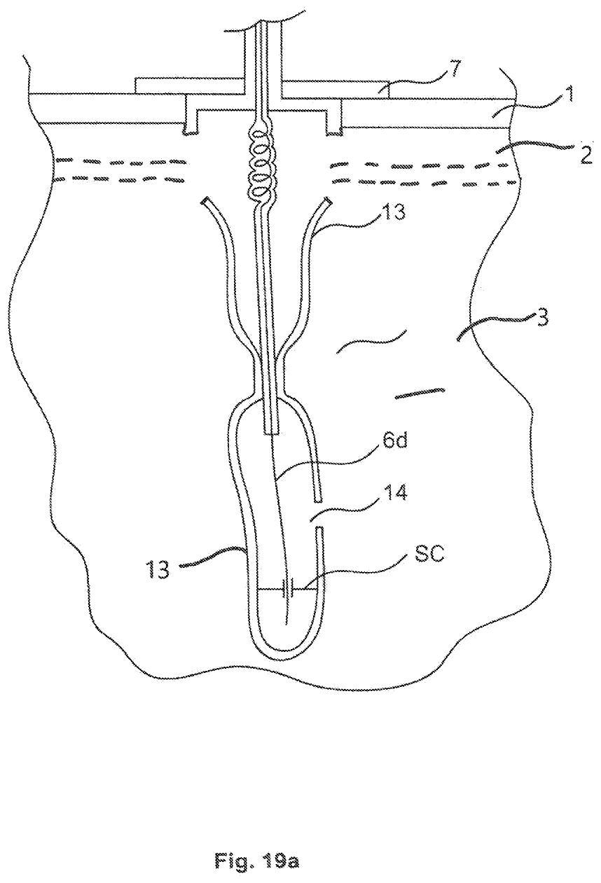

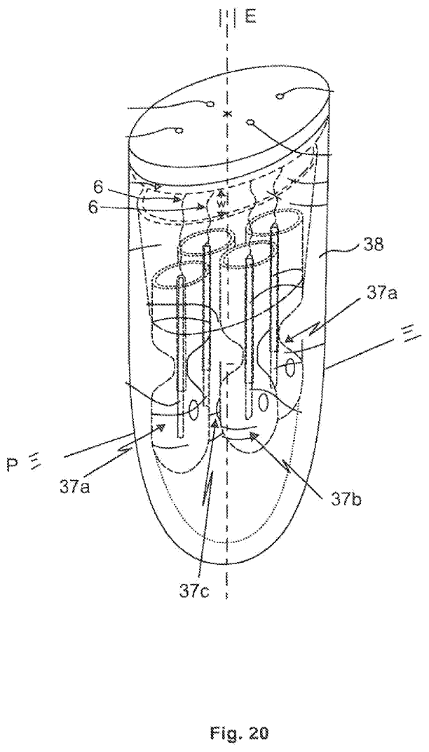

FIG. 20 illustrate an array of four microelectrodes (37a), (37b), (37c), (37d). The microelectrodes are embedded in an array matrix (38).

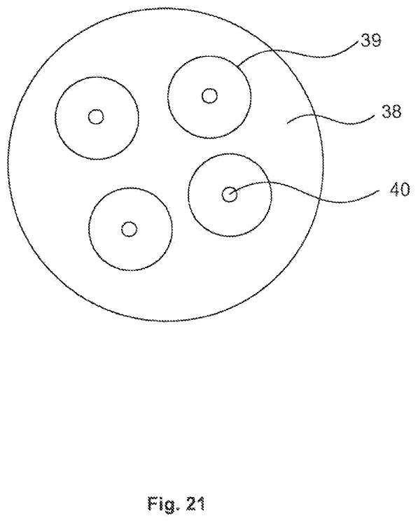

FIG. 21 illustrates a cross-section of an array at allocation P showing the array matrix (38), a casing encapsulating a distal compartment (39) and a distal non-insulated portion of the conductive element (40).

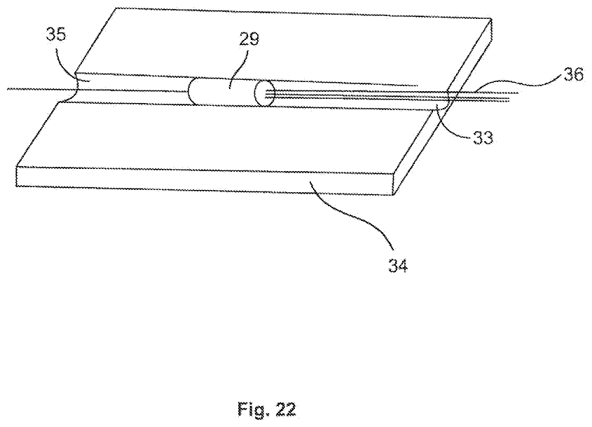

FIG. 22 illustrates a manufacturing step in the manufacturing of a microelectrode with a first structural element (29) of a different material than the casing. The first structural element is positioned around the proximal insulated portion of a conductive element (36) and placed within one first half of a mold (34) of silicone. The second half of the mold properly is positioned with regard to the first half of the mold. Before casting the proximal and distal matrices it is preferred to position the element centrally with respect to the mold.

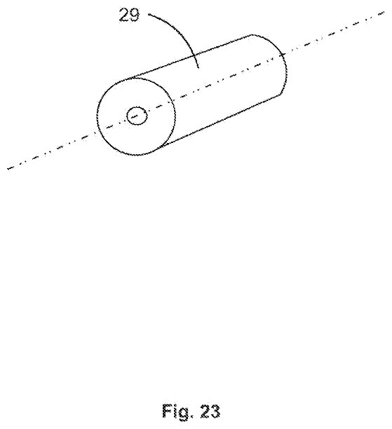

FIG. 23 shows a perspective view of first structural component (29) and the central axis as a dashed line.

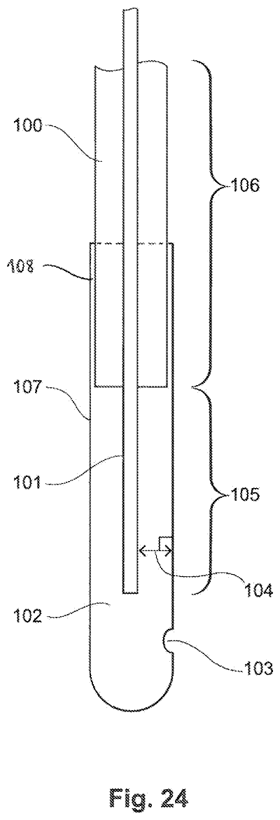

FIG. 24 shows a variant of the microelectrode comprising a conductive element (101). A proximal portion of the conductive element (106) is insulated with an electrically insulating non-degradable material (100) while a distal portion of the conductive element is non-insulated (105). A casing (107) of flexible electrically insulating non-degradable material encapsulates the non-insulated portion of the conductive element (105) forming a distal chamber (102). The casing of the distal chamber comprises an opening (103). The inner radial extension of the casing is such that it provides a void/lumen (108) between the casing and the insulated portion of the conductive element (106) for enabling an axial movement of the casing with respect to the conductive element. The numeral (104) visualizes what is meant by the perpendicular distance between the non-insulated portion of the conductive element (105) and the opening (103).

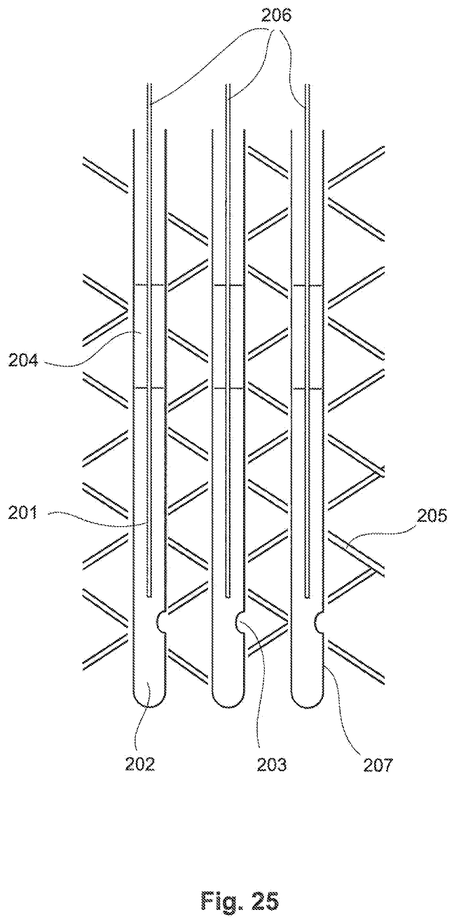

FIG. 25 presents a first array of microelectrodes attached to one another by micro- or nano-fibers (205). The conductive element (206), first structural components (204), casing (207), distal chambers (202), and opening in the casing of the distal chambers (203) are shown. For reasons of simplicity, the insulation of the conductive elements are not indicated. An array of microelectrodes attached to one another by micro- or nano-fibers preferably having an extension providing a patch. The individual microelectrodes may be arranged essentially parallel in essentially one plane combined forming an array exhibiting a patch-like global extension. This type of array may be applied for monitoring and/or stimulating spinal nervous tissue.

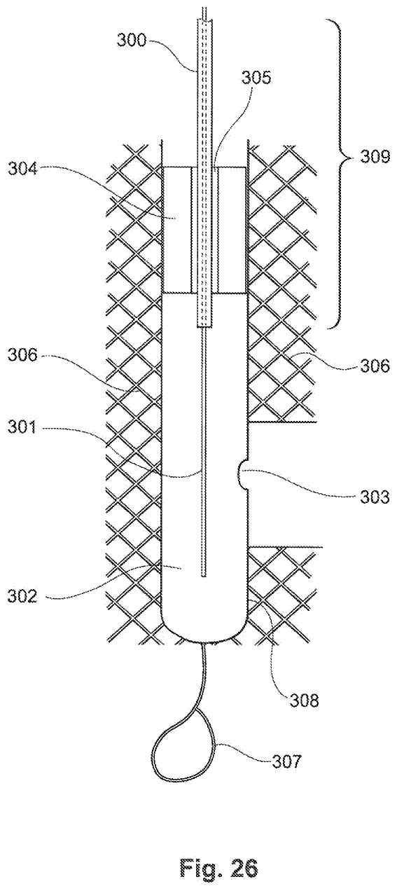

FIG. 26 shows a variant of a microelectrode comprising an engagement element (307). The casing (308) exhibits a net of micro- or nano-fibers (306) which preferably are adhesively attached to the external surface of the casing. The micro- or nano-fibers increase friction of the casing with respect to the surrounding soft tissue. FIG. 26 also presents a void/lumen (annular channel) (305) between the first structural component (304) and the insulation (300) around the conductive element (301) and surrounding the insulated portion of the proximal conductive element (309). For reasons of clarity the dimensions of the void are exaggerated. The engagement element is configured to reversibly engage with an elongated rigid pin such as a needle (not shown). The pin is further configured to insert the microelectrode into the soft tissue or placing the microelectrode adjacent to soft tissue.

1-60. (canceled)

61. A microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber having at least one opening providing (after implantation) a fluidic electrically conductive bridge between the non-insulated portion of the conductive element and the soft tissue enabling an exchange of ions between the distal chamber and the tissue, wherein the at least one opening is useful for recording and stimulation of electrically excitable cells, wherein the casing comprises a first structural component in which the electrically insulated portion of the conductive element can slide in an axial direction.

62. The microelectrode according to claim 61, wherein the first structural component partitions the casing (envelope) into a distal chamber and a proximal compartment.

63. The microelectrode according to claim 61, wherein at least part of the electrically insulated portion is localized within the distal chamber.

64. The microelectrode according to claim 61, wherein a lumen/void (enabling axial movements) is provided between the first structural component and the electrically insulated portion of the conductive element

65. The microelectrode according to claim 61, wherein the innermost material(s) of the casing and/or the first structural components and/or the outermost material of the proximal electrically insulated portion of the element is/are (each) selected to reduce friction.

66. The microelectrode according to claim 61, wherein the distal chamber comprises a second structural component configured to reducing radial movement of the non-insulated portion of the conductive element relative to the distal casing, while also being configured to enable an axial movement of the non-isolated conductive element with respect to the second structural component.

67. The microelectrode according to claim 61, wherein the perpendicular distance between the non-insulated portion of the conductive element and the at least one opening in the casing of the distal chamber remains essentially the same during axial movements of the casing relative to the conductive element, optionally the perpendicular distance not changing more than 20%.

68. The microelectrode according to claim 61, wherein the at least one opening has an area of at least about 1 μm2.

69. The microelectrode according to claim 61, wherein the distal chamber comprises a plurality of openings in the distal casing.

70. The microelectrode according to claim 61, wherein the distal portion of the casing of the distal chamber has a three-dimensional shape narrowing in distal direction such as a spherical shape.

71. The microelectrode according to claim 61, wherein a proximal portion of the distal chamber narrows down, preferably exhibiting an annular form forming the first structural component, in which the electrically insulated portion of the conductive element can slide in an axial direction.

72. The microelectrode according to claim 61, wherein the friction between the casing and the adjacent soft tissue is higher that the friction between the innermost material of the casing and/or the first structural component and/or the outermost material of the proximal electrically insulated portion of the element.

73. The microelectrode according to claim 61, wherein the outermost material and/or outermost surface structure of the casing is selected to increase friction against the soft tissue.

74. The microelectrode according to claim 61, wherein the casing of the distal chamber comprises an engagement element configured to reversibly engage with an elongated rigid pin such as a needle, the pin being configured to insert the microelectrode into the soft tissue or placing the microelectrode adjacent to soft tissue, the engagement element comprised at the distal portion of the distal chamber.

75. The microelectrode according to claim 74, wherein the engagement element is a loop or net comprising micro- or nanofibers

76. The microelectrode according to claim 74, wherein the engagement element is degradable in body fluids.

77. The microelectrode according to claim 61, wherein the casing comprises means for increasing friction between the casing and the adjacent soft tissue.

78. The microelectrode according to claim 77, wherein the means for increasing friction is selected from micro- or nano-fibers attached to the outermost surface of the casing.

79. The microelectrode according to claim 61, wherein a void/lumen between the first structural element and the outermost layer of the proximal electrically insulated portion of the conductive element comprises a composition facilitating the movement of the first structural element with respect to the outermost layer, particularly a composition comprising any one of lipids, hyaluronic acid, silicones (such as silicone oil or silicone grease) and a polymer of monosaccharides such as glucose and combinations thereof.

80. The microelectrode according to claim 61, wherein the casing has a rotationally symmetric shape, suitably cylindrical shape.

81. The microelectrode according to claim 62, wherein the diameter of the proximal compartment widens in a proximal direction.

82. The microelectrode according to claim 61, wherein the distal chamber and optionally the proximal compartment comprises at least one biologically active substance such as a pharmaceutically active substance.

83. The microelectrode according to claim 61, wherein the conductive element extending proximally of the proximal compartment is of a material or of materials different from that or those of the conductive element disposed in the proximal and distal compartments.

84. The microelectrode according to claim 61, wherein the electrically insulating material of the casing is a biocompatible, non-degradable flexible polymeric material, particularly a biocompatible, flexible polymeric selected from polyurethanes, polyethylenes, polymers with a backbone comprising benzene (e.g. parylenes such as Parylene C and Parylene M), and polymers based on the polymerization of tetrafluoroethylene and flexible inorganic materials (such as glass or glass-like materials)

85. The microelectrode according to claim 61, wherein the distal chamber, and optionally the proximal compartment, comprises a biocompatible material dissolvable or degradable in aqueous body fluids and providing structural support to the microelectrode when dry.

86. A microelectrode probe comprising a microelectrode as defined by claim 61, wherein the distal chamber, and optionally the proximal compartment, comprise(s) a biocompatible material providing structural support to the probe when dry for insertion into soft tissue, wherein the biocompatible material is dissolvable or degradable in aqueous body fluids.

87. The microelectrode according to claim 61, wherein the microelectrode or microelectrode probe is embedded in an embedding matrix of a biocompatible material providing sufficient rigidity to the probe when dry for insertion into soft tissue and dissolvable or degradable in aqueous body fluids.

88. The microelectrode according to claim 61, further comprising an element holder, the electrically conductive element extending (in proximal direction) through the element holder, the holder configured to be secured to a tissue different from the soft tissue, in particular osseous or connective tissue.

89. The microelectrode according to claim 61, wherein the electrically conductive element is in electrical engagement with an apparatus for registration of biological signals and stimulation of soft tissue.

90. The microelectrode according to claim 61, wherein the biocompatible matrix-materials are selected from carbohydrate-based materials, protein-based materials, and non-natural polymeric materials, and mixtures thereof.

91. A first array of microelectrodes according to claim 61, wherein the microelectrodes are adhesively attached to micro or nanofibers.

92. The first array according to claim 91, wherein the microfibers are degradable.

93. A second array of microelectrodes according to claim 61 partially or entirely embedded in an array matrix of a biocompatible material providing sufficient rigidity to the array when dry for insertion into soft tissue and dissolvable or degradable in aqueous body fluids.

94. The microelectrode according to claim 61, wherein the biocompatible dissolvable or degradable materials are selected from carbohydrate-based materials, protein-based materials, and non-natural polymeric materials, and mixtures thereof.

95. The second array according to claim 93, further comprising an array cover.

96. The second array according to claim 95, wherein the array matrix extends to the distal face of the array cover.

97. The second array according to claim 93, further comprising an array casing of a flexible, non-degradable material embracing a part of the array matrix.

98. The second array according to claim 97, embedded in an outer array matrix of a biocompatible material which is solid when dry and dissolvable or degradable in aqueous body fluids.

99. The second array according to claim 98, wherein the biocompatible materials are selected from carbohydrate-based materials, protein-based materials, and non-natural polymeric materials, and mixtures.

100. A method for manufacturing the microelectrode according to claim 61, comprising:
providing an elongated electrically conductive element;
covering a proximal portion of the element with an electrically insulating layer thereby providing a proximal electrically insulated portion and a distal non-insulated portion of the conductive element;
forming a distal matrix dissolvable or degradable in aqueous body fluids extending axially around, and optionally extending in a distal direction from the distal non-insulated portion of the conductive element;
applying a sliding facilitating composition to a section of the insulated element proximally with respect to the distal matrix and distally with respect to an optional proximal matrix wherein the sliding facilitating composition is facilitating the axial movement of a first layer of electrically insulating non-degradable material of the conductive element, said medium optionally providing for a sufficient void/lumen between the insulating layer of the conductive element and first layer of electrically insulating non-degradable material;
optionally forming a proximal matrix extending axially around at least part of the proximal electrically insulated portion of the conductive element;
covering the distal matrix and at least part of the proximal electrically insulated portion of the conductive element with a first layer of electrically insulating non-degradable material, thereby providing a casing encapsulating the distal non-insulated portion of the element forming a distal chamber and a first structural element;
cutting part of the non-insulated portion of the conductive element and first layer of electrically insulating non-degradable material near the distal end of the distal matrix (distal end of the distal chamber) comprising the distal non-insulated portion of the electrically conductive element, thereby providing a distal opening of the distal compartment;
applying a further distal tip matrix distally to the distal opening; and
covering the tip matrix and at least part of the first layer with a second layer of electrically insulating non-degradable material, thereby forming a distal end cap part forming part of the casing of the distal chamber,
wherein the distal and optionally proximal matrices provide structural support to the microelectrode or probe when dry for insertion into soft tissue, and
wherein at least one opening through the first layer and optionally second layer of the casing of the distal chamber is provided.

101. A method for manufacturing the microelectrode according to claim 62, comprising:
providing an elongated electrically conductive element;
covering a proximal portion of the element with an electrically insulating layer thereby providing a proximal electrically insulated portion and a distal non-insulated portion of the conductive element;
forming a distal matrix dissolvable or degradable in aqueous body fluid extending axially around, and optionally extending in a distal direction from the distal non-insulated portion of the conductive element;
forming a proximal matrix extending axially around at least part of the proximal electrically insulated portion of the conductive element and thereby forming an intermediate section of the insulated conductive element with an axial extension, the intermediate section positioned proximally to the distal matrix and distally to the proximal matrix not covered by the distal and proximal matrices;
applying a thin (up to about 5 μm) layer of a first intermediate matrix and/or sliding facilitating composition to the intermediate section of the insulated element facilitating the axial movement of a first layer of electrically insulating non-degradable material with respect to the insulating layer of the conducting element, said first intermediate matrix and/or composition providing for a sufficient void/lumen (annular channel) between the electrically insulated portion of the conductive element and the first layer of electrically insulating non-degradable material;
covering distal, proximal matrices and the intermediate section of the proximal electrically insulated portion of the element, the intermediate section comprising an intermediate matrix and/or sliding facilitating composition, with a first layer of electrically insulating non-degradable material, thereby providing a casing comprising a distal chamber, a first structural element and a proximal compartment;
optionally providing a second intermediate matrix on the first layer of electrically insulating non-degradable material in the constriction in radial direction of the first layer between the distal chamber and proximal compartment;
cutting part of the distal non-insulated portion of the electrically conductive element and the first layer of electrically insulating material near the distal end of the distal matrix (distal end of the distal chamber), thereby providing a distal opening of the distal chamber;
applying a further distal tip matrix distally to the distal opening;
covering the distal tip matrix and at least part of the first layer with a second layer of electrically insulating material thereby forming a distal end cap forming part of the casing of the distal chamber; and
removing the first layer and optionally second layer at a circumferential annular zone of the proximal matrix,
wherein the distal matrix, distal tip matrix, proximal matrix and optionally first and second intermediate matrices are of a biocompatible material providing sufficient rigidity to the probe when dry for insertion into soft tissue and dissolvable or degradable in aqueous body fluids, and
wherein at least one opening is provided through the first and optionally second layers of the casing of the distal chamber.

102. A method for manufacturing the microelectrode according to claim 61, comprising:
providing an elongated electrically conductive element;
covering a proximal portion of the element with an electrically insulating layer thereby providing a proximal electrically insulated portion and a distal non-insulated portion of the element;
providing a first structural element configured to enable an axial movement with respect to the proximal electrically insulated portion of the conductive element;
positioning the first structural element around the proximal electrically insulated portion of the conductive element, suitably at a certain axial distance from the distal non-insulated portion of the conductive element;
optionally applying a proximal matrix dissolvable or degradable in aqueous body fluids around the proximal electrically insulated portion of the conductive element, the proximal matrix extending from the proximal face of the first structural element in proximal direction;
applying a distal matrix dissolvable or degradable in aqueous body fluids around the distal non-insulated portion of the element extending from the distal face of the first structural element in distal direction, and extending in a distal direction from the distal non-insulated portion of the conductive element, suitably up to several millimeters; and
applying a first layer of electrically insulating non-degradable material on the optional proximal and distal matrices and the circumference of the first structural element, thereby forming a casing comprising a distal chamber and a proximal compartment,
wherein at least one opening is provided through the first layer of the casing of the distal chamber.

103. The method according to claim 100, wherein the proximal matrix widens in a proximal direction.
